(19) United States
(12) Patent Application Publication
(10) Pub. No.: US 2023/0248689 A1
(43) Pub. Date: Aug. 10, 2023

(54) PHARMACEUTICAL FORMULATIONS OF GRISEOFULVIN FOR LONG-TERM OCULAR DELIVERY

(71) Applicants: Purdue Research Foundation, West Lafayette, IN (US); THE TRUSTEES OF INDIANA UNIVERSITY, Bloomington, IN (US); Dr. Reddy's Laboratories Limited, Hyderabad (IN)

(72) Inventors: Yoon Yeo, West Lafayette, IN (US); Dhawal Chobisa, West Lafayette, IN (US); Timothy W. Corson, Fishers, IN (US)

(73) Assignees: PURDUE RESEARCH FOUNDATION, West Lafayette, IN (US); THE TRUSTEES OF INDIANA UNIVERSITY, Bloomington, IN (US)

(21) Appl. No.: 18/012,589

(22) PCT Filed: Jun. 24, 2021
(86) PCT No.: PCT/US21/38792

§ 371 Date: Dec. 22, 2022

Related U.S. Application Data
(60) Provisional application No. 63/044,405, filed on Jun. 26, 2020.

(51) Int. Cl. A61K 31/343; A61K 9/51

(52) U.S. Cl. CPC A61K 31/343; A61K 9/5153

(57) ABSTRACT

The present invention relates to pharmaceutical formulations for long-term ocular delivery of the active agents. The present invention further provides the long-term ocular delivery of griseofulvin with specific in vitro release profile. These formulations are used for the treatment of neovascular eye diseases and age-related macular degeneration (AMD). In particular, the present invention provides microparticles & nanoparticles of griseofulvin for the long-term ocular delivery and methods of preparation of such formulations.

### CROSS-REFERENCE TO RELATED APPLICATIONS

The present U.S. patent application relates to and claims the priority benefit of U.S. Provisional Patent Application Ser. No. 63/044,405, filed Jun. 26, 2020, the contents of which are hereby incorporated by reference in their entirety into this present disclosure.

### TECHNICAL FIELD

The present invention relates to pharmaceutical formulations for long-term ocular delivery of the active agents. In particular, the present invention provides pharmaceutical formulations of griseofulvin in the form of microparticles or nanoparticles for long-term ocular delivery and methods of preparation of such formulations. These formulations are used for the treatment of neovascular eye diseases and age-related macular degeneration (AMD).

### BACKGROUND

This section introduces aspects that may help facilitate a better understanding of the disclosure. Accordingly, these statements are to be read in this light and are not to be understood as admissions about what is or is not prior art.

Age-related macular degeneration (AMD) is the leading cause of irreversible vision loss in elderly people. It is a chronic, progressive disease that destroys the sharp central vision. AMD is one of the most common disease of retina. It is classified as atrophic or dry-AMD and neovascular or wet-AMD. Dry-AMD involves drusen formation (lipid and protein deposits under the retina) and progressive retinal pigment epithelium atrophy, ultimately converting to wet AMD. Wet-AMD is characterized by subretinal and sub-RPE proliferation of abnormal blood vessels that leak fluid, blood, and lipids.

Established treatment modalities for AMD include thermal laser photocoagulation or photodynamic therapy in conjunction with verteporfin. More recently, anti-vascular endothelial growth factor therapies such as pegaptanib, ranibizumab, aflibercept, and bevacizumab have shown success in slowing and even reversing vision loss in some age-related macular degeneration patients. But the significant acute, systemic side effects (e.g., non-ocular hemorrhage, myocardial infarction, and stroke) indicate that these therapies can act outside the eye, even when delivered intravitreally. Blinding intraocular side effects are also possible and the long-term risks of these drugs are still unclear. Moreover, because they are biologics, the cost-benefit ratios of these drugs are unfavorable.

Further this current treatment of wet-AMD with intravitreal injection of antivascular endothelial growth factor drugs such as Pegaptanib, Ranibizumab, Aflibercept, Brolucizumab, many patients developed resistance. Recently identified was an alternative therapeutic target, i.e., ferrochelatase (FECH), as a new target protein for wet-AMD treatment. Ferrochelatase (FECH) is an enzyme that catalyzes the terminal step of biosynthesis of heme, and an essential protein for angiogenesis. FECH can be inhibited by N-methyl protoporphyrin (NMPP), which is formed by the cytochrome P450-mediated bioconversion of Griseofulvin (GRF).

U.S. Ser. No. 10/752,901 patent describes the methods of inhibiting ocular angiogenesis by administering several different agents that inhibit ferrochelatase, one among them is griseofulvin. The chemical name of griseofulvin, is (2S,6′R)-7-chloro-2′,4,6-trimethoxy-6′-methyl-3H-spiro[benzofuran-2,1′-cyclohexan]-2′-ene-3,4′-dione:

Griseofulvin:

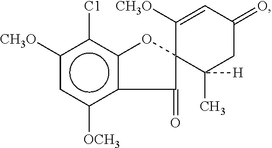

(2S,6′R)-7-chloro-2′,4,6-trimethoxy-6′-methyl-3H-spiro[benzofuran-2,1′-cyclohexan]-2′-ene-3,4′-dione; CAS NO. 126-07-8.

Currently griseofulvin is commercially available only in the form of oral dosage forms (GRIS-PEG® Tablets, 125 mg & 250 mg) as an anti-fungal therapy.

WO2019213076A1 patent application describes a triazolopyrimidinone, or a derivative thereof, or a pharmaceutically acceptable salt thereof can function as ferrochelatase (FECH) inhibitor used for the treatment of neovascular diseases, such as neovascular eye diseases. FECH has been identified as a mediator of ocular neovascularization. Neovascularization is a key pathological determinant of a number of major, blinding eye diseases including, but not limited to, retinopathy of prematurity, wet age-related macular degeneration (AMD), and proliferative diabetic retinopathy.

Importantly, the neovascularization associated with wet AMD has features in common with a variety of other eye diseases, collectively known as neovascular eye diseases, in which new blood vessels grow in the tissues of the eye. These diseases include, but are not limited to, retinopathy of prematurity (ROP), proliferative diabetic retinopathy (PDR), diabetic retinopathy, wet age-related macular degeneration (AMD) pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and the like.

Nonetheless, there remains a need in the art for new administration regimens for angiogenic eye disorders, especially those which allow for less frequent dosing while maintaining a high level of efficacy. None of the above references discloses any specific pharmaceutical formulation for long-term ocular delivery of griseofulvin.

Hence there is a critical unmet need for the development of pharmaceutical formulations for long-term ocular delivery of griseofulvin and process of preparing such formulations which is simple, feasible and commercially viable.

### SUMMARY OF THE INVENTION

In some illustrative embodiments, the present invention relates to a method to manufacturing a drug-loaded liposome comprising the steps of:

The present invention relates to pharmaceutical formulations for long-term ocular delivery of the active agents. In particular, the present invention provides pharmaceutical formulations of griseofulvin in the form of microparticles or nanoparticles for long-term ocular delivery and methods of preparation of such formulations. These formulations are used for the treatment of age-related macular degeneration (AMD).

Aspects of the present invention provide pharmaceutical formulation for long-term ocular delivery of griseofulvin or its pharmaceutically acceptable salts, derivatives thereof, methods of preparation of such pharmaceutical formulation and its use for the treatment for AMD.

In one embodiment, the present invention provides pharmaceutical formulation for ocular administration comprising griseofulvin or its pharmaceutically acceptable salts, derivatives thereof, and at least one biocompatible polymer, wherein the formulation exhibits long-term release of griseofulvin for a period of about 1 month to about 6 months, or about 1 month to about 3 months, or about 1 month. According to any embodiment of the present invention, griseofulvin is in the form of free base, salt, solvate, complex, co-crystal or combinations thereof.

In another embodiment, the pharmaceutical formulation of the present invention exhibits an in vitro griseofulvin release profile of
less than about 40% release within 1 day;
about 40% to about 70% release within 10 days; and
more than about 70% release within 30 days.

In another embodiment, the pharmaceutical formulation of the present invention exhibits an in vitro griseofulvin release profile of
less than about 30% release within 1 day;
about 40% to about 70% release within 10 days; and
more than about 80% release within 30 days.

In another embodiment, the pharmaceutical formulation of the present invention exhibits an in vitro griseofulvin release profile of
less than about 20% release within 1 day,
about 40% to about 70% of release within 10 days,
about 70% to about 85% release within 20 days; and
more than about 85% release within 30 days

In another embodiment, the pharmaceutical formulation of the present invention exhibits an in vitro griseofulvin release profile of
less than about 20% release within 1 day,
about 40% to about 70% of release within 10 days,
about 75% to about 85% release within 20 days; and
more than about 85% release within 30 days.

In some embodiments, 100% of the griseofulvin is released within about 40 days. In other embodiments, 100% of the griseofulvin is released within about 35 days. In other embodiments, 100% of the griseofulvin is released within about 30 days.

In one embodiment, the biocompatible polymer according to the present invention is selected from the group comprising of poly(lactides), poly(glycolides), poly(lactide-co-glycolides), poly(lactic acid)s, poly(glycolic acid)s, poly(lactic acid-co-glycolic acids)s, polycaprolactones, polycarbonates, polyesteramides, polyanhydrides, poly(amino acids), polyorthoesters, polycyanoacrylates, poly(p-dioxanone)s, poly(alkylene oxalate)s, biodegradable polyurethanes, blends and copolymers thereof. In another embodiment, the biocompatible polymer is poly(lactic-co-glycolic acid) (PLGA).

In another embodiment, the PLGA of the present invention has an average molecular weight range from about 1,000 to about 150,000 Daltons, or about 5,000 to about 1,00,000 Daltons, or about 25,000 to about 75,000 Daltons. In one embodiment of the present invention, the average molecular weight of the PLGA ranges from about 40,000 to about 75,000 Daltons. In another embodiment, the average molecular weight of the PLGA ranges from about 50,000 to about 70, 000 Daltons.

In another embodiment, the PLGA of the present invention has a molar ratio of lactic acid to glycolic acid range from about 90:10 to about 10:90 or about 85:15 to about 15:85 or about 75:25 to about 25:75. In one embodiment, the molar ratio of the PLGA ranges from about 60:40 to about 40:60. In another embodiment, the molar ratio of the PLGA is about 50:50.

In another embodiment of the present invention the PLGA concentration in the pharmaceutical formulation is about 70% to about 99% or about 75% to about 95% by weight relative to the total weight of the formulation. In another embodiment, the PLGA concentration in the pharmaceutical formulation is about 85% to about 95% by weight relative to the total weight of the formulation.

In another embodiment, the pharmaceutical formulations of the present invention further comprising a release modifier. Release modifier according to any embodiment is selected from the group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran. In another embodiment, the release modifier according to the present invention comprising magnesium hydroxide or magnesium phosphate.

In another embodiment of the present invention, the release modifier concentration is about 0.25% to about 30% by weight relative to the total weight of the formulation or about 0.5 to about 20% by weight or about 1% to about 15% by weight relative to the total weight of the formulation. In another embodiment, the release modifier concentration is less than about 10%, or less than about 5% by weight relative to the total weight of the formulation. In another embodiment, the release modifier concentration is about 2% by weight relative to the total weight of the formulation.

In another embodiment, the pharmaceutical formulation according to the present invention, wherein griseofulvin is in the form of microparticles (MPs) or nanoparticles (NPs) or combinations thereof.

In an embodiment, microparticles or nanoparticles of the present invention has the encapsulation efficiency ranges from about 10% to about 90% or about 20% to about 80% or about 25% to about 75%. In another embodiment of the present invention, the span value of griseofulvin microparticles is about 0.5 to about 5 or about 1 to about 4. In another embodiment of the present invention, the polydispersity index of griseofulvin nanoparticles ranges about 0.1 to about 1.0. In another embodiment of the present invention, the porosity of the microparticles or nanoparticles is less than about 50% or less than about 30% or less than about 20% or less than about 10%. In another embodiment, the porosity of the microparticles or nanoparticles is about 5%.

In another embodiment, the present disclosure relates to the pharmaceutical formulations as disclosed herein, wherein the weight ratio of griseofulvin to biocompatible polymer in the microparticles or nanoparticles ranges from about 1:3 to about 1:50 or about 1:5 to about 1:40. In another embodiment, the weight ratio of griseofulvin to biocompatible polymer in the microparticles or nanoparticles ranges from about 1:5 to about 1:30.

In another embodiment, the present disclosure relates to a pharmaceutical formulation as disclosed herein, wherein griseofulvin is in the form of microparticles. In another embodiment, the mean particle size of griseofulvin microparticles ranges from about 5 μm to about 100 μm. In another embodiment, the mean particle size of griseofulvin microparticles ranges from about 5 μm to about 50 μm.

In another embodiment, the present disclosure relates to the pharmaceutical formulations as disclosed herein, wherein griseofulvin is in the form of nanoparticles. In another embodiment, the mean particle size of griseofulvin nanoparticles ranges from about 10 nm to about 1000 nm. In another embodiment, the mean particle size of griseofulvin nanoparticles ranges from about 10 nm to about 1000 nm. In another embodiment, the mean particle size of griseofulvin nanoparticles ranges from about 100 nm to about 600 nm.

In another embodiment of the present invention, a pharmaceutical formulation for long-term ocular administration of griseofulvin comprises
griseofulvin or its salts, derivatives thereof at the concentration of about 0.5% to about 25% by weight of the formulation;
a biocompatible polymer at the concentration of about 70% to about 99% by weight of the formulation; and
a release modifier at the concentration of about 0.25% to about 30% by weight of the formulation.

According to one embodiment of the present invention, the biocompatible polymer is PLGA and release modifier is magnesium hydroxide or magnesium phosphate.

In another embodiment of the present invention, a pharmaceutical formulation for long-term ocular administration of griseofulvin comprises
griseofulvin or its salts, derivatives thereof at the concentration of about 0.5% to about 25% by weight of the formulation;
poly(lactic-co-glycolic acid) polymer at the concentration of about 70% to about 99% by weight of the composition and magnesium hydroxide or magnesium phosphate at the concentration of about 0.25% to about 30% by weight of the composition;
wherein the formulation exhibits an in vitro griseofulvin release profile of less than about 40% release within 1 day;
about 40% to about 70% release within 10 days; and more than about 70% release within 30 days.

Another aspect of the present invention provides the process of preparation of the pharmaceutical formulations of griseofulvin for long-term ocular delivery. In one embodiment, the pharmaceutical formulations of the present invention are prepared by the emulsion technique. In another embodiment, the pharmaceutical formulations of the present invention are prepared by the single emulsion technique or double emulsion technique. In another embodiment, the microparticles of the present invention are prepared by the double emulsion method. In another embodiment, the nanoparticles of the present invention are prepared by the single emulsion technique.

In another embodiment, a process of preparation of the pharmaceutical formulation of griseofulvin for long-term ocular administration comprises
preparing a solution by dissolving griseofulvin and a biocompatible polymer in a suitable solvent;
mixing the griseofulvin-biocompatible polymer solution with an emulsifying agent to form a dispersion;
preparing an emulsion by mixing the dispersion with water and/or aqueous solvent system comprising an emulsifier; and
removal of the solvents by using a suitable drying method.

In another embodiment, a process of preparing the pharmaceutical formulation of griseofulvin for long-term ocular administration comprises
preparing a primary emulsion by mixing a) griseofulvin-biocompatible polymer solution prepared by dissolving griseofulvin and a biocompatible polymer in suitable organic solvent system and b) solution or suspension of a release modifier prepared by dissolving/dispersing the release modifier in water and/or aqueous solvent system containing an emulsifier;
preparing a secondary emulsion by mixing the primary emulsion with a suitable emulsifying agent; and removing the solvents by using a suitable drying method.

In another embodiment, the biocompatible polymer is PLGA. In another embodiment, a release modifier is magnesium hydroxide or magnesium phosphate and the amount of release modifier ranges from about 0.25% to about 30 wt % relative to the total weight of the formulation.

Suitable solvents used in the process of preparation of the solution of griseofulvin and a biocompatible polymer is selected from group comprising dichloromethane (DCM), acetone, dimethyl sulfoxide (DMSO), dimethylformamide, acetonitrile, tetrahydrofuran, ethyl acetate, chloroform, acetone, hexafluoroisopropanol etc.; Suitable emulsifying agents according to the process of preparation of the present invention are selected from the group comprising PVA and/or other surfactants that optionally can be included are one or more of the following: non-ionic surfactants (such as Poloxamers, Tweens), anionic surfactants (such as sodium oleate, sodium stearate or sodium lauryl sulfate), gelatin, polyvinylpyrrolidone, carboxymethyl cellulose and its derivatives.

In another embodiment, the pharmaceutical formulations of the present invention are administered by different routes of ocular administration. In one embodiment, the pharmaceutical formulations of the present invention, wherein the formulation is administered by intravitreal injection.

In another embodiment, the pharmaceutical formulations of the present invention are used for the treatment of neovascular eye diseases and age-related macular degeneration. In another embodiment, the pharmaceutical formulations of the present invention are used for the treatment of wet-age related macular degeneration (Wet-AMD).

### BRIEF DESCRIPTION OF THE DRAWINGS

These and other features, aspects and advantages of the present invention will be better understood with reference to the following figures, descriptions and claims.

FIG. 1 shows in vitro drug release of the Griseofulvin PLGA nanoparticles with varied LA:GA ratio of PLGA. 50:50 PLGA (Example 2A) and 85:15 PLGA (Example 2B).

FIG. 2 shows in vitro drug release profiles of the Griseofulvin PLGA nanoparticles obtained with PLGA molecular weight 4 kDa (Example 3A), 54-69 kDa (Example 3B) & 100-120 kDa (Example 3C).

FIG. 3 depicts anti-proliferative effect of Griseofulvin PLGA nanoparticles of Example 3B in comparison with the free drug griseofulvin (Free GRF) on human retinal endothelial cells (HRECs)

FIG. 4 shows the porosity of the Griseofulvin PLGA microparticles with varying amounts of magnesium hydroxide.

FIG. 5 shows in vitro drug release from Griseofulvin PLGA microparticles with varying amounts of magnesium hydroxide.

FIG. 6 shows the long-term proliferation assay of Griseofulvin PLGA microparticles on HRECs. Numbers in the box indicates the time-averaged proliferation inhibition index (PII), calculated as Σ AUC/time), where AUC is the area under the curve.

FIG. 7 demonstrates in vitro drug release from Griseofulvin PLGA microparticles with varying amounts of magnesium phosphate.

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the term “derivative” or “derivatives” refers to a structurally similar compound that retains sufficient functional attributes of the given compound.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Furthermore, the term “pharmaceutically-acceptable excipient” as used herein means one or more compatible solid or liquid fillers, diluents or encapsulating substances which are suitable for administration into a human or other mammal. The term “excipient” denotes an organic or inorganic ingredient, natural or synthetic, with which the active ingredient-containing microparticles or nanoparticles are combined to facilitate the application. The components of the pharmaceutical formulations are capable of being co-mingled with the components of the present disclosure (e.g., the active agent, the biocompatible polymer), and with each other, in a manner such that there is no interaction that substantially impairs the desired pharmaceutical efficacy. Pharmaceutically acceptable excipients further means a non-toxic material that is compatible with a biological system such as a cell, cell culture, tissue, or organism. The characteristics of the carrier depend on the route of administration. Physiologically and pharmaceutically acceptable excipients include diluents, buffering agents, stabilizers, bulking agents, preservatives, tonicity adjusting agents, surfactants, antioxidants, chelating agents, suspending agents etc. and other materials which are well known in the art. Pharmaceutically acceptable carriers suitable for parenteral administration such as ophthalmic, subcutaneous, intravenous, intramuscular, or other type of administrations also are well known.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intravitreal, suprachoroidal, subretinal, intraperitoneal, epidural, intraurethral, intratarsal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” or “subject” disclosed herein includes human and non-human animals such as companion animals (dogs and cats and the like) and livestock animals. Livestock animals are animals raised for food production. The patient to be treated is preferably a mammal, in particular a human being.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits a long-term release of griseofulvin for a period of about 1 month to about 6 months.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits long-term release of griseofulvin for a period of about 1 month to about 3 months.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits long-term release of griseofulvin for a period of about 1 month.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits an in vitro griseofulvin release profile of
less than about 40% release within 1 day; about 40% to about 70% release within 10 days; and more than about 70% release within 30 days.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits an in vitro griseofulvin release profile of
less than about 30% release within 1 day; about 40% to about 70% release within 10 days; and more than about 80% release within 30 days.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation exhibits an in vitro griseofulvin release profile of
less than about 20% release within 1 day; about 40% to about 70% release within 10 days; about 70% to about 85% release within 20 days; and more than about 85% release within 30 days.

In some illustrative embodiments, this disclosure relates to a pharmaceutical formulation for ocular administration comprising griseofulvin or its pharmaceutically acceptable salts, derivatives thereof, and at least one biocompatible polymer, wherein the biocompatible polymer is selected from the group comprising of poly(lactides), poly(glycolides), poly(lactide-co-glycolides), poly(lactic acid)s, poly(glycolic acid)s, poly(lactic acid-co-glycolic acids)s, polycaprolactones, polycarbonates, polyesteramides, polyanhydrides, poly(amino acids), polyorthoesters, polycyanoacrylates, poly(p-dioxanone)s, poly(alkylene oxalate)s, biodegradable polyurethanes, blends and copolymers thereof.

In some illustrative embodiments, this disclosure relates to a pharmaceutical formulation for ocular administration comprising griseofulvin or its pharmaceutically acceptable salts, derivatives thereof, and at least one biocompatible polymer, wherein the biocompatible polymer is poly(lactic-co-glycolic acid) polymer.

In some illustrative embodiments, this disclosure relates to a pharmaceutical formulation for ocular administration comprising griseofulvin or its pharmaceutically acceptable salts, derivatives thereof, and at least one biocompatible polymer, wherein the poly(lactic-co-glycolic acid) polymer has an average molecular weight ranges from about 1,000 to about 150,000 Daltons.

In some illustrative embodiments, this disclosure relates to a pharmaceutical formulation for ocular administration comprising griseofulvin or its pharmaceutically acceptable salts, derivatives thereof, and at least one biocompatible polymer, wherein the poly(lactic-co-glycolic acid) polymer has an average molecular weight ranges from about 40,000 to about 75,000 Daltons.

In some illustrative embodiments, this disclosure relates to a pharmaceutical formulation for ocular administration comprising griseofulvin or its pharmaceutically acceptable salts, derivatives thereof, and at least one biocompatible polymer, wherein the poly (lactic-co-glycolic acid) polymer has a molar ratio of lactic acid to glycolic acid ranges from about 90:10 to about 10:90.

In some illustrative embodiments, this disclosure relates to a pharmaceutical formulation for ocular administration comprising griseofulvin or its pharmaceutically acceptable salts, derivatives thereof, and at least one biocompatible polymer, wherein the poly (lactic-co-glycolic acid) polymer has a molar ratio of lactic acid to glycolic acid ranges from about 60:40 to about 40:60.

In some illustrative embodiments, this disclosure relates to a pharmaceutical formulation for ocular administration comprising griseofulvin or its pharmaceutically acceptable salts, derivatives thereof, and at least one biocompatible polymer, wherein the poly (lactic-co-glycolic acid) polymer concentration is about 70% to about 99% by weight relative to the total weight of the formulation.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation further comprises a release modifier selected from the group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation further comprises a release modifier of magnesium hydroxide or magnesium phosphate.

In some illustrative embodiments, this disclosure relates to a pharmaceutical formulation for ocular administration comprising griseofulvin or its pharmaceutically acceptable salts, derivatives thereof, and at least one biocompatible polymer, wherein the formulation further comprises a release modifier having a concentration of about 0.25% to about 30% by weight relative to the total weight of the formulation.

In some illustrative embodiments, this disclosure relates to a pharmaceutical formulation for ocular administration comprising griseofulvin or its pharmaceutically acceptable salts, derivatives thereof, and at least one biocompatible polymer, wherein griseofulvin is in the form of microparticles or nanoparticles or a combination thereof.

In some illustrative embodiments, this disclosure relates to a pharmaceutical formulation for ocular administration comprising griseofulvin or its pharmaceutically acceptable salts, derivatives thereof, and at least one biocompatible polymer, wherein griseofulvin is in the form of microparticles.

In some illustrative embodiments, this disclosure relates to a pharmaceutical formulation for ocular administration comprising griseofulvin or its pharmaceutically acceptable salts, derivatives thereof, and at least one biocompatible polymer, wherein the mean particle size of griseofulvin microparticles ranges from about 5 μm to about 100 μm.

In some illustrative embodiments, this disclosure relates to a pharmaceutical formulation for ocular administration comprising griseofulvin or its pharmaceutically acceptable salts, derivatives thereof, and at least one biocompatible polymer, wherein the mean particle size of griseofulvin microparticles ranges from about 5 μm to about 50 μm.

In some illustrative embodiments, this disclosure relates to a pharmaceutical formulation for ocular administration comprising griseofulvin or its pharmaceutically acceptable salts, derivatives thereof, and at least one biocompatible polymer, wherein the encapsulation efficiency of griseofulvin microparticles ranges from about 10 to about 90%.

In some illustrative embodiments, this disclosure relates to a pharmaceutical formulation for ocular administration comprising griseofulvin or its pharmaceutically acceptable salts, derivatives thereof, and at least one biocompatible polymer, wherein the porosity of griseofulvin microparticles is less than about 50%.

In some illustrative embodiments, this disclosure relates to a pharmaceutical formulation for ocular administration comprising griseofulvin or its pharmaceutically acceptable salts, derivatives thereof, and at least one biocompatible polymer, wherein the span value of griseofulvin microparticles is about 0.5 to about 5.

In some illustrative embodiments, this disclosure relates to a pharmaceutical formulation for ocular administration comprising griseofulvin or its pharmaceutically acceptable salts, derivatives thereof, and at least one biocompatible polymer, wherein the weight ratio of griseofulvin to biocompatible polymer in the microparticles ranges from about 1:3 to about 1:50.

Yet in some other illustrative embodiments, this disclosure relates to a pharmaceutical formulation for a long-term ocular administration of griseofulvin comprising
a) griseofulvin or its salts, derivatives thereof at the concentration of about 0.5% to about 25% by weight of the formulation;
b) a biocompatible polymer at the concentration of about 70% to about 99% by weight of the formulation; and
c) a release modifier at the concentration of about 0.25% to about 30% by weight of the formulation.

In some other illustrative embodiments, this disclosure relates to a pharmaceutical formulation for a long-term ocular administration of griseofulvin as disclosed herein, wherein the biocompatible polymer is poly(lactic-co-glycolic acid) polymer and release modifier is magnesium hydroxide.

In some other illustrative embodiments, this disclosure relates to a pharmaceutical formulation for a long-term ocular administration of griseofulvin as disclosed herein, wherein the biocompatible polymer is poly(lactic-co-glycolic acid) polymer and release modifier is magnesium phosphate.

In some other illustrative embodiments, this disclosure relates to a pharmaceutical formulation for a long-term ocular administration of griseofulvin as disclosed herein, wherein the formulation exhibits long-term release of griseofulvin for a period of about 1 month to about 3 months.

In some other illustrative embodiments, this disclosure relates to a pharmaceutical formulation for a long-term ocular administration of griseofulvin as disclosed herein, wherein the formulation exhibits long-term release of griseofulvin for a period of about 1 month.

In some other illustrative embodiments, this disclosure relates to a pharmaceutical formulation for a long-term ocular administration of griseofulvin as disclosed herein, wherein the formulation exhibits an in vitro griseofulvin release profile of
a) less than about 40% release within 1 day;
b) about 40% to about 70% release within 10 days; and
c) more than about 70% release within 30 days.

In some other illustrative embodiments, this disclosure relates to a pharmaceutical formulation for a long-term ocular administration of griseofulvin as disclosed herein, wherein the formulation exhibits an in vitro griseofulvin release profile of
a) less than about 20% release within 1 day;
b) about 40% to about 70% of release within 10 days;
c) about 70% to about 85% release within 20 days; and
d) more than about 85% release within 30 days.

In some other illustrative embodiments, this disclosure relates to a pharmaceutical formulation for a long-term ocular administration of griseofulvin as disclosed herein, wherein griseofulvin is in the form of nanoparticles.

In some other illustrative embodiments, this disclosure relates to a pharmaceutical formulation for a long-term ocular administration of griseofulvin as disclosed herein, wherein the mean particle size of griseofulvin nanoparticles ranges from about 10 nm to about 1000 nm.

In some other illustrative embodiments, this disclosure relates to a pharmaceutical formulation for a long-term ocular administration of griseofulvin as disclosed herein, wherein the mean particle size of griseofulvin nanoparticles ranges from about 100 nm to about 600 nm.

In some other illustrative embodiments, this disclosure relates to a pharmaceutical formulation for a long-term ocular administration of griseofulvin as disclosed herein, wherein the encapsulation efficiency of griseofulvin nanoparticles ranges from about 10% to about 90%.

In some other illustrative embodiments, this disclosure relates to a pharmaceutical formulation for a long-term ocular administration of griseofulvin as disclosed herein, wherein the porosity of griseofulvin nanoparticles is less than about 50%.

In some other illustrative embodiments, this disclosure relates to a pharmaceutical formulation for a long-term ocular administration of griseofulvin as disclosed herein, wherein the polydispersity index of griseofulvin nanoparticles is about 0.1 to about 1.0.

In some other illustrative embodiments, this disclosure relates to a pharmaceutical formulation for a long-term ocular administration of griseofulvin as disclosed herein, wherein the weight ratio of griseofulvin to biocompatible polymer in the nanoparticles ranges from about 1:3 to about 1:50.

In some other illustrative embodiments, this disclosure relates to a process of preparing the pharmaceutical formulation of griseofulvin for long-term ocular administration comprising
a) preparing a solution by dissolving griseofulvin and a biocompatible polymer in a suitable solvent;
b) mixing the griseofulvin-biocompatible polymer solution with an emulsifying agent to form a dispersion;
c) preparing an emulsion by mixing the dispersion with water and/or aqueous solvent system containing an emulsifier; and
d) removal of the solvents by using a suitable drying method.

In some other illustrative embodiments, this disclosure relates to a process of preparing the pharmaceutical formulation of griseofulvin for long-term ocular administration comprising
a) preparing primary emulsion by mixing a) griseofulvin-biocompatible polymer solution prepared by dissolving griseofulvin and a biocompatible polymer in a suitable organic solvent system and b) solution or suspension of a release modifier prepared by dissolving/dispersing a release modifier in water and/or aqueous solvent system containing an emulsifier;
b) preparing secondary emulsion by mixing the primary emulsion with an aqueous solution containing a suitable emulsifying agent; and
c) removal of the solvents by using a suitable drying method.

In some other illustrative embodiments, this disclosure relates to a process of preparing the pharmaceutical formulation of griseofulvin for long-term ocular administration as disclosed herein, wherein the biocompatible polymer is poly(lactic-co-glycolic acid) polymer and release modifier is selected from group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran.

In some other illustrative embodiments, this disclosure relates to a process of preparing the pharmaceutical formulation of griseofulvin for long-term ocular administration as disclosed herein, wherein the suitable solvent is selected from the group comprising of dichloromethane, acetonitrile, tetrahydrofuran, ethylacetate, chloroform, acetone, hexafluoroisopropanl.

In some other illustrative embodiments, this disclosure relates to a process of preparing the pharmaceutical formulation of griseofulvin for long-term ocular administration as disclosed herein, wherein the suitable emulsifying agent is selected from the group comprising of polyvinyl alcohol (PVA), non-ionic surfactants (such as Poloxamers, Tweens), anionic surfactants (such as sodium oleate, sodium stearate or sodium lauryl sulfate), gelatin, polyvinylpyrrolidone, carboxymethyl cellulose and its derivatives.

In some other illustrative embodiments, this disclosure relates to the pharmaceutical formulation of griseofulvin as disclosed herein, wherein the formulation is administered by different routes of ocular administration selected from intravitreal injection, suprachoroidal injection, subretinal injection, intraocular injection, periocular injection, intra bulbar injection, intracameral injection, sub-tenon injection, subconjunctival injection, ocular insert, or an implant.

In some other illustrative embodiments, this disclosure relates to a pharmaceutical formulation of griseofulvin as disclosed herein, wherein the formulation is administered by intravitreal injection.

In some other illustrative embodiments, this disclosure relates to a pharmaceutical formulation of griseofulvin as disclosed herein, wherein the formulation is used for the treatment of age-related macular degeneration.

In some other illustrative embodiments, this disclosure relates to a pharmaceutical formulation of griseofulvin as disclosed herein, wherein the formulation is used for treatment of wet-age related macular degeneration.

In some other illustrative embodiments, this disclosure relates to a method for the treatment of an eye disease of a patient comprising the step of administrating a therapeutically effective amount of a pharmaceutical formulation as disclosed herein.

In some other illustrative embodiments, this disclosure relates to a method for the treatment of an eye disease of a patient comprising the step of administrating a therapeutically effective amount of a pharmaceutical formulation manufactured according to the process as disclosed herein.

In some other illustrative embodiments, this disclosure relates to a method for the treatment of an eye disease of a patient comprising the step of administrating a therapeutically effective amount of a pharmaceutical formulation as disclosed herein, wherein said eye diseases are selected from the group consisting of retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

The pharmaceutical compositions disclosed herein may include the inhibitors and, optionally, additional therapeutic agents and pharmaceutical carriers. Together with the methods of the present disclosure, said pharmaceutical compositions may also be administered to a subset of subjects in need of treatment for neovascular eye disease, including retinopathy of prematurity (ROP), proliferative diabetic retinopathy (PDR), diabetic retinopathy, wet age-related macular degeneration (AMD) pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and the like. Some subjects that are in specific need of treatment for ocular neovascular disease may include subjects who are susceptible to, or at elevated risk of, experiencing ocular neovascular disease (e.g., retinopathy of prematurity, diabetic retinopathy, “wet” age-related macular degeneration, etc.), and the like. Subjects may be susceptible to, or at elevated risk of, experiencing ocular neovascular diseases due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

Aspects of the present invention provides pharmaceutical formulations for long-term ocular delivery of the active agents. In particular, the present invention provides pharmaceutical formulations of griseofulvin in the form of microparticles or nanoparticles for long-term ocular delivery and methods of preparation of such formulations. These formulations are used for the treatment of age-related macular degeneration (AMD).

Aspects of the present invention provides pharmaceutical formulation for long-term ocular delivery of griseofulvin or its pharmaceutically acceptable salts, derivatives thereof, methods of preparation of pharmaceutical formulation and its treatment for AMD.

Active agents of the present invention are any agent used for the treatment of Age-related macular degeneration. Active agent of the present invention includes but not limited to griseofulvin, Pegaptanib, Ranibizumab, Aflibercept, Brolucizumab, Conbercept, or combinations thereof. In one embodiment, an active agent of the present invention is griseofulvin or its pharmaceutically acceptable salts, derivatives thereof.

According to any embodiment of the present invention, griseofulvin is in the form of free base, salt, solvate, complex, co-crystal or combinations thereof. Complexes might be formed by addition of griseofulvin and inorganic compounds. Suitable salts include inorganic or organic acids or polymeric acids. The acid used to form the pharmaceutically acceptable salt of the active agent has a pKa less than 5. The acids suitable to form the pharmaceutically acceptable salt of active agents may be selected from, but not limited to, the group consisting of hydrochloric acid, hydrobromic acid, nitric acid, chromic acid, sulfuric acid, methanesulfonic acid, trifluromethane sulfonic acid, trichloroacetic acid, dichloroacetic acid, bromoacetic acid, chloroacetic acid, cyanoacetic acid, 2-chloropropanoic acid, 2-oxobutanoic acid, 2-chlorobutanoic acid, 4-cyanobutanoic acid, pamoic acid, perchloric acid, phosphoric acid, hydrogen iodide, acetic acid, 2,2-dichloroacetic acid, adipic acid, alginic acid, L-ascorbic acid, L-aspartic acid, benzenesulfonic acid, benzoic acid, 4-acetamido benzoic acid, (+)-camphoric acid, (+)-camphor-10-sulfonic acid, capric acid, (decanoic acid), caproic acid (hexanoic acid), caprilic acid (octanoic acid)carbonic acid, cinnamic acid, citric acid, cyclamic acid, decanoic acid, dodecylsulfuric acid, ethane-1,2-disufonic acid, ethanesulfonic acid, 2-hydroxy-ethanesulfonic acid, formic acid, fumaric acid, galactic acid, gentisic acid, D-glucoheptonic acid, D-gluconic acid, D-glucuronic acid, glutamic acid, glutaric acid, 2-oxo-glutaric acid, glycerophosphoric acid, glycolic acid, hippuric acid, isobutyric acid, DL-lactic acid, lactobionic acid, lauric acid, maleic acid, (−)-L-malic acid, malonic acid, DL-mandelic acid, muric acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, embonic acid, proprionic acid, (−)-L-pyroglutamic acid, salicyclic acid, 4-amino-salicylic acid, sebacic acid, stearic acid, succinic acid, (+)-L-tartaric acid, thiocyanic acid, p-toluenesulfonic acid, undecylenic acid. The selection of the suitable acids is well-known to those of skill in the art.

Age-related macular degeneration (AMD) is a posterior eye disease that affects a person's central vision. This is a common cause of blindness. The AMD can be “atrophic” or “dry” macular degeneration and “neovascular” or “Wet” macular degeneration.

FECH has been identified as a new target protein for wet-AMD treatment. Ferrochelatase (FECH) is an enzyme that catalyzes the terminal step of biosynthesis of heme, and is an essential protein for angiogenesis. FECH can be inhibited by N-methyl protoporphyrin (NMPP), which is formed by the cytochrome P450-mediated bioconversion of griseofulvin (GRF).

In one embodiment of the present invention, pharmaceutical formulations for long-term ocular delivery comprising griseofulvin or its pharmaceutically acceptable salts, derivatives thereof are used for the treatment of AMD, in particular wet-AMD.

“Long-term release” term has been used here in the description is interchangeably considering different modified release systems such as prolonged release, sustained release, extended release, controlled release, modified release, delayed release, and repeat action. And the “long-term release” in the context of the present invention relates to a release of said agent over a given, prolonged period of time. In one embodiment, the long-term release encompasses the release of the active agent for a period of one week to several months up to about 1 year or for a period of about 1 month to about 6 months or for a period of about 1 month to about 3 months. In another embodiment, the long-term release encompasses the release of the active agent for a period of about 1 month.

The “long-term release” in the context of the present invention, relates to in vitro release in specific dissolution medium or in vivo release when administered by ocular route.

In an embodiment, the pharmaceutical formulations according to the present invention are administered by ocular routes. Different delivery types for ophthalmic or ocular routes include but not limited to Eye drops, Ointment, Hydrogels including in-situ forming gels, Emulsions including microemulsions and nano emulsions, Ophthalmic inserts/ocular inserts, contact lenses, intraocular injections, Novel forms such as periocular and intravitreal injection, intra bulbar injections, suprachoroidal injection, Intracameral injection, sub-tenon injection, subconjunctival injection, including systemic and topical administration. In an embodiment, the pharmaceutical formulations are administered by intravitreal injection, implants and ocular inserts. In one embodiment, the pharmaceutical formulations of the present invention are administered by intravitreal injection.

As used herein, the term “initial burst release” defined as the amount of active agent that has been released within 1 day. As used herein, the term “about” modifies a particular value by referring to a range equal to the particular value plus or minus 0.1% to 20%.

In one embodiment, the present invention provides pharmaceutical formulation for ocular administration comprising griseofulvin or its pharmaceutically acceptable salts, derivatives thereof and at least one biocompatible polymer, wherein the formulation exhibits long-term release of griseofulvin for a period of about 1 month to about 6 months, or for a period of about 1 month to about 3 months, or for a period of about 1 month.

In another embodiment, the pharmaceutical formulation of the present invention exhibits an in vitro griseofulvin release profile of
less than about 30% release within 1 month,
about 35% to about 65% release within 3 months, and
more than about 80% release within 6 months.

In another embodiment, the pharmaceutical formulation of the present invention exhibits an in vitro griseofulvin release profile of
less than about 40% release within 1 day,
about 40% to about 70% release within 10 days, and
more than about 70% release within 30 days.

In another embodiment, the pharmaceutical formulation of the present invention exhibits an in vitro griseofulvin release profile of
less than about 30% release within 1 day,
about 40% to about 70% release within 10 days, and
more than about 80% release within 30 days.

In another embodiment, the pharmaceutical formulation of the present invention exhibits an in vitro griseofulvin release profile of
less than about 20% release within 1 day,
about 40% to about 70% of release within 15 days,
about 70% to about 80% release within 20 days, and
more than about 85% release within 30 days.

In another embodiment, the pharmaceutical formulation of the present invention exhibits an in vitro griseofulvin release profile of
less than about 20% release within 1 day,
about 40 to about 70% of release within 10 days,
about 75 to about 85% release within 20 days, and
more than about 90% release within 30 days.

In some embodiments, 100% of the griseofulvin is released within about 40 days. In other embodiments, 100% of the griseofulvin is released within about 35 days. In other embodiments, 100% of the griseofulvin is released within about 30 days.

According to any embodiment of the present invention, the initial burst release of the compositions is less than about 40%, or less than about 30%. In another embodiment of the present invention, the initial burst release of the compositions is less than about 20% or less than about 15% by weight relative to the total weight of the formulation.

Biocompatible polymers suitable according to the present invention are either biodegradable or non-biodegradable polymers or blends or copolymers thereof. A polymer is biocompatible if the polymer and any degradation products of the polymer are non-toxic to the recipient and also possess no significant deleterious or untoward effects on the recipient's body, such as an immunological reaction at the injection site. “Biodegradable”, as defined herein, means the composition will degrade or erode in vivo to form smaller chemical species. Degradation can result, for example, by enzymatic, chemical and physical processes. The term “biodegradable polymer” as used herein is meant to include any biocompatible and/or biodegradable synthetic and natural polymers that can be used in vivo. Generally, the biodegradable polymer of the present invention is polyester. These polyesters may be a linear polymer, or a branched or star polymer, or a mixture of a linear polymer and a branched and/or star polymer. In one embodiment, the biocompatible polymer of the present invention is lactate-based polymer.

The lactate-based polymer of the present invention includes homopolymers of lactic acid or lactide monomers (poly(lactic acid) or polylactide, PLA), and copolymers of lactic acid (or lactide) with other monomers (for example, glycolic acid (or glycolide) (poly(lactide-co-glycolide), PLG or PLGA) and the like). The lactate-based polymer may have the same end groups, i.e., all the end groups are the same, such as ester, or hydroxyl or carboxylic acid. The lactate-based polymer may have mixed end groups of ester, hydroxyl, and/or carboxylic acid. The lactate-based polymer can have a diol core with end hydroxyl groups. Similarly, the lactate-based polymer may have a triol or polyol core, such as glucose, with end hydroxyl groups. The lactate-based polymer may have one end group as an ester and the other end with a hydroxyl group or carboxylic acid group. The lactate-based polymer may also have one end hydroxyl group and the other end with a carboxylic acid or an ester, or vice versa.

In one embodiment, the suitable biocompatible, biodegradable polymers include but not limited to poly(lactides), poly(glycolides), poly(lactide-co-glycolides), poly(lactic acid)s, poly(glycolic acid)s, polycarbonates, polyesteramides, polyanhydrides, poly(amino acids), polyorthoesters, poly(dioxanone)s, poly(alkylene alkylate)s, copolymers or polyethylene glycol and polyorthoester, biodegradable polyurethane, blends, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl fluoride, poly(vinyl imidazole), chlorosulphonate polyolefins, polyethylene oxide, blends, and copolymers thereof.

In another embodiment, the pharmaceutical formulation according to the present invention, wherein griseofulvin is in the form of microparticles (MPs) or nanoparticles (NPs) or combinations thereof.

In the field of long-term release microparticle or nanoparticle compositions, it is known in the art that various factors or parameters affect the release rate. These factors are the type of polymer, concentration of the polymer and its molecular weight, copolymer composition, the nature of the excipients added to the composition, method of manufacturing, type of the drug etc.

In one embodiment, the biocompatible polymer of the present invention comprises poly(lactic-co-glycolic acid) polymer (PLGA).

These polymers are available in a variety of molecular weights, and the appropriate molecular weight to provide the desired release rate for the active agent is readily determined by one of skill in the art. In one embodiment, PLGA used according to the invention having a molecular weight range from about 1,000 and about 150,000 Daltons or about 5,000 to about 100,000 Daltons, or about 25,000 to about 75,000 Daltons. In one embodiment, the average molecular weight of the PLGA ranges from about 40,000 to about 75, 000 Daltons. In another embodiment, the average molecular weight of the PLGA ranges from about 50,000 to about 70,000 Daltons.

In an embodiment, the poly (lactic-co-glycolic acid) polymer as per the present invention has a molar ratio of lactic acid to glycolic acid ranges from about 90:10 to about 10:90. In another embodiment the said ratio is ranges from about 85:15 to about 15:85. In another embodiment the molar ratio of lactic acid to glycolic acid ranges from about 75:25 to about 25:75. In another embodiment the molar ratio lactic acid to glycolic acid ranges from about 60:40 to about 40:60. In another embodiment, the molar ratio of lactic acid to glycolic acid as per the present invention is ranges from about 55:45 to about 45:55. In another embodiment, the molar ratio of lactic acid to glycolic acid as per the present invention is about 50:50.

In another embodiment of the present invention the PLGA concentration in the pharmaceutical formulation is about 70% to about 99% or about 75% to about 95% by weight relative to the total weight of the formulation. In another embodiment, the PLGA concentration in the pharmaceutical formulation is about 85% to about 95% to the total weight of the formulation

The microparticle or nanoparticle formulations of the present invention may contain drug loading in a range of about 0.01% to about 40% by weight relative to the total weight of the formulation. In one embodiment, the drug loading efficiency is about 0.5% to about 20% by weight relative to the total weight of the formulation. In another embodiment, the drug loading efficiency ranges from about 5% to about 15% by weight relative to the total weight of the formulation. In general, the optimal drug loading efficiency depends upon the period of release desired and the potency of the active agent. The drug loading efficiency is defined as the mass ratio of the drug to drug-loaded nanoparticles or microparticles.

In an embodiment, microparticles or nanoparticles of the pharmaceutical formulations of the present invention has an encapsulation efficiency ranges from about 10% to about 90% or about 20% to about 80% or about 25% to about 75%. The Encapsulation efficiency as mentioned here is defined as the percentage of drug that is successfully entrapped into the microparticles or nanoparticles. According to the embodiment of the present invention, the encapsulation efficiency is calculated as described below:

$$Encapsulation efficiency ( E E \% ) = \frac{Actual loading efficiency ( L E , \% )}{Theoretical loading efficiency ( T L E , \% )} * 100$$

$$Loading efficiency ( L E \% ) = \frac{Weight of griseofulvin measured}{Weight of microparticles analyzed} * 100$$

In another embodiment of the present invention, the porosity of the microparticles or nanoparticles is less than about 50% or less than about 30% or less than about 20% or less than about 10%. In another embodiment of the present invention, the porosity of the microparticles or nanoparticles is about 5%. The porosity as mentioned here is defined as the as the ratio of the total pore volume to the apparent volume of the microparticles or nanoparticles, excluding interparticle voids. The porosity of particles is measured by the image analysis by SEM. According to the SEM images, the drug release from microparticles or nanoparticles is driven by the initially existing pores as well as the increasing porosity during the incubation. A higher initial porosity leads to the high burst drug release, because of a larger effective surface area for drug diffusion. The later phase drug release is governed by an increase in porosity during the incubation. In both phases, a higher porosity facilitates the infusion of release medium into the microparticles or nanoparticles and dissolution of drug located in the core.

In another embodiment of the present invention, the span value of griseofulvin microparticles is about 0.5 to about 5 or about 1 to about 4. In another embodiment of the present invention, the polydispersity index (PDI) of griseofulvin nanoparticles ranges about 0.1 to about 1.0 or about 0.1 to about 0.8. The PDI/Span value indicates whether the dispersion of nanoparticles or microparticles is monodisperse/polydisperse or having narrow/broad particle size distribution.

PDI is the ratio of mass average molecular mass to the number average molecular mass. The span is defined as (D₉₀−D₁₀)/D₅₀. The particle size, which is represented in terms of D₁₀, D₅₀, D₉₀. For example, D₅₀ means 50% of particles are having size less than this value. The particle size and particle size distribution were estimated in triplicate through a Master sizer 3000 (Malvern Panalytical, UK; Zetasizer).

In vitro release of the microparticles or nanoparticles according to the pharmaceutical formulations of the present invention is evaluated in phosphate buffered saline (pH 7.4) with 0.2% Tween 80 (PBST) at 37° C. In one embodiment, the in vitro release of the formulation is carried out by the following process: i.e. Microparticles or nanoparticles were prepared such that the total griseofulvin concentration was below one-third of the saturation solubility to satisfy the sink condition. 15 mL microparticle or nanoparticle suspension was filled in a 15 mL Falcon tube and rotated vertically at 10 rpm. At predetermined time intervals, 1 mL suspension was sampled, centrifuged to separate the supernatant and analyzed by HPLC for determination of griseofulvin concentration. Alternatively, any suitable dissolution apparatus can be used for in vitro release study.

The formulations of the present invention, i.e. microparticles (MPs) or nanoparticles (NPs) of the griseofulvin (GRF) were tested for in vitro cell study, in particular long-term proliferation assay. This study provides an estimation of in vivo effect of griseofulvin formulations. To compare the long-term antiproliferative effect on human retinal endothelial cells (HREC), time averaged proliferation inhibition index (PII) was calculated (Σ AUC/time) from the plot of % proliferation at each time vs. time. The higher the PII indicating the prolonged anti-proliferative effects on HREC.

In another embodiment, the pharmaceutical formulations of the present invention wherein the weight ratio of griseofulvin to biocompatible polymer in the microparticles or nanoparticles ranges from about 1:3 to about 1:50 or about 1:5 to about 1:40. In another embodiment, the weight ratio of griseofulvin to biocompatible polymer in the microparticles or nanoparticles ranges from about 1:5 to about 1:30 or 1:5 to about 1:20.

In other embodiment, the pharmaceutical formulation according to the present invention, wherein griseofulvin is in the form of microparticles. In another embodiment, the mean particle size of griseofulvin microparticles ranges from about 5 μm to about 100 μm. In another embodiment, the mean particle size of griseofulvin microparticles ranges from about 5 μm to about 50 μm.

In another embodiment, the pharmaceutical formulation according to the present invention, wherein griseofulvin is in the form of nanoparticles. In another embodiment, the mean particle size of griseofulvin nanoparticles ranges from about 10 nm to about 1000 nm. In another embodiment, the mean particle size of griseofulvin nanoparticles ranges from about 10 nm to about 1000 nm preferably about 100 nm to about 600 nm.

According to an embodiment of the present invention, a Master Sizer 3000 (Malvern Panalytical, UK) was used to measure the particle size and the particle size distribution of PLGA loaded microparticles, and a Zetasizer Nano ZS90 for nanoparticles. Wet dispersion method was used to analyze the microparticle size. According to one embodiment the wet dispersion method was carried out by the following method of approximately 10 mg of microparticles were dispersed in 500 μL of water and sonicated. Dispersion was added drop wise into the measuring unit until desired obscurity (2%-10%) was obtained.

In one embodiment of the present invention provides pharmaceutical formulation for ocular administration comprising griseofulvin or its pharmaceutically acceptable salts, derivatives thereof, and at least one biocompatible polymer. In other embodiment of present invention provides pharmaceutical formulation for ocular administration comprising griseofulvin or its pharmaceutically acceptable salts, derivatives thereof, and at least one biocompatible polymer, further comprising a release modifier.

In another embodiment, the release modifier according to the present invention is selected from the group comprising magnesium hydroxide, magnesium carbonate, magnesium phosphate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran or combinations thereof. In another embodiment, the release modifier according to the present invention comprising magnesium hydroxide or magnesium phosphate.

In another embodiment of the present invention, the release modifier concentration is about 0.25% to about 30% by weight relative to the total weight of the formulation or about 0.5 to about 20% by weight or about 1% to about 15% by weight relative to the total weight of the formulation. In another embodiment, the release modifier concentration is less than about 10%, or less than about 5% by weight relative to the total weight of the formulation. In another embodiment, the release modifier concentration is about 2% by weight relative to the total weight of the formulation.

PLA or PLGA-based systems generate acidic products in degrading matrices, reducing the pH inside of the polymeric structure (microclimate pH, pH). The acidifying PLA or PLGA matrices can cause significant issues in release control, stability and activity of the loaded active agents. With the recognition of damaging effects of acidifying pH of PLA- and PLGA systems on drug stability and release kinetics control, various formulation approaches have been employed to counteract the acidifying pH, aiming to neutralize by basic excipients and/or delay the acidification by facilitating the diffusion of acidic products.

When carefully selected according to the unique mechanisms of these release modifiers, which can help control drug stability and release kinetics without making drastic changes in the encapsulated drugs or polymeric matrices. These release modifiers were coencapsulated in PLGA systems to neutralize the acidifying pH and protect the stability of encapsulated drugs. The rationale of this approach is that the bases neutralize the acidic pH, reducing the acid-catalyzed polymer degradation, and also react with low molecular weight polymer degradants to form salts, creating osmotic pressure to enhance water influx.

In another embodiment of the present invention, a pharmaceutical formulation for long-term ocular administration of griseofulvin comprises
griseofulvin or its salts, derivatives thereof at the concentration of about 0.5% to about 25% by weight of the formulation;
a biocompatible polymer at the concentration of about 70% to about 99% by weight of the formulation; and
a release modifier at the concentration of about 0.25% to about 30% by weight of the formulation.

In another embodiment of the present invention, a pharmaceutical formulation for long-term ocular administration of griseofulvin comprises
griseofulvin or its salts, derivatives thereof at the concentration of about 0.5% to about 10% by weight of the formulation;
poly(lactic-co-glycolic acid) polymer at the concentration of about 70% to about 99% by weight of the formulation; and
magnesium hydroxide at the concentration of about 0.25% to about 30% by weight of the formulation.
   wherein the formulation exhibits an in vitro griseofulvin release profile of
less than about 40% release within 1 day;
about 40% to about 70% release within 10 days; and
more than about 70% release within 30 days.

Another aspect of the present invention provides the process of preparation of the pharmaceutical formulations for long-term ocular delivery of griseofulvin or pharmaceutically acceptable salts or derivatives thereof.

There are number of techniques for the preparation of microparticles or nanoparticles, especially microparticles and nanoparticles manufactured from PLGA. The most widely used techniques both in lab scale and for commercial productions include phase separation/coacervation technique, spray drying and single or double emulsion/solvent evaporation technique (PDA J Pharm Sci and Tech 2008, 62 125-154; Microencapsulation Methods and Industrial Applications Second Edition). The following four examples are for explanation purpose only, not intended in any way to limit the scope of this present disclosure.

1. In Emulsion technique, Oil-in-water (o/w) and water-in-oil-water (w/o/w) are the two hydrous techniques representing, respectively the single and double emulsion formation during microparticle or nanoparticles preparation.

This single emulsion technique involves the formation of an oil in water (O/W) in which polymer and drug are dissolved together in an appropriate solvent. At present, halogenated solvents with a low boiling point such as dichloromethane, chloroform, hexafluoro-isopropanol and/or non-halogenated solvents like ethyl acetate, isopropanol, methyl ethyl ketone, acetone and benzyl alcohol are preferred, otherwise mixed solvent are used. This solution represents the oil phase (O), and it is added by sonication or homogenization to the water phase, consisting of water and a surfactant or emulsifying agent as polyvinyl alcohol (PVA), polyethylene glycol sorbitan monolaurate (Tween), sorbitan monooleate (Span), sodium dodecyl sulfate (SDS) to form the final emulsion. The mature microparticles or nanoparticles are formed during the elimination of the solvent by evaporation that can be facilitated by a continuous stirring or using an under-pressure solvent drying system or by solvent extraction.

In the double emulsion technique, active agents are dissolved in an organic solvent (e.g., alcohol) or in an aqueous solution and then mixed or emulsified with an organic solution (non-miscible with water) of the polymer to form a solution or water-in-oil (w/o) emulsion, respectively. Dichloromethane serves as organic solvent for the PLGA and the o/w primary emulsion is formed using either high-shear homogenization or ultrasonication. This primary emulsion is then rapidly transferred to an excess of aqueous medium containing a stabilizer, usually polyvinyl alcohol (PVA). Again, homogenization or intensive stirring is necessary to initially form a w/o/w double emulsion. Subsequent removal (by evaporation) of organic solvent by heat, vacuum, or both results in phase separation of polymer and core to produce micro- or nanoparticles. Instead of solvent evaporation, solvent extraction with large quantity of water with or without a stabilizer can also be undertaken to yield required microparticles or nanoparticles.

2. In phase-separation or coacervation technique, an aqueous solution of active agent is emulsified or alternatively the active agent is dispersed in solid form into solution containing dichloromethane and PLGA. Silicone oil is added to this dispersion at a defined rate, reducing solubility of polymer in its solvent. The polymer-rich liquid phase (coacervate) encapsulates the dispersed active agent, and embryonic micro/nanoparticles are subjected to hardening process by means of hardening agent to get the hardened micro/nanoparticles. Filtering & washing them with suitable solvent to remove the residual solvent is followed by drying to get the finished micro/nanoparticles.

3. In spray-drying technique a polymer is dissolved in a volatile organic solvent such as dichloromethane or acetone. The active agent is suspended as solid or emulsified as aqueous solution in this organic solution by homogenization. After that, the resulting dispersion is atomized through a (heated) nozzle into a heated air flow. The organic solvent evaporates, thereby forming micro/nanoparticles. These micro/nanoparticles are collected in a cyclone separator. For the complete removal of the organic solvent, a vacuum-drying or Lyophilization step can follow downstream. The internal structure of the resulting polymeric microparticles depends on the solubility of the active agent in the polymer prior to spray-drying leading to the formation of reservoir or matrix type products. When the initial dispersion is solution, the final product obtained following spray drying is matrix or monolithic type, that is, polymer particles with dissolved or dispersed nature of the active agent. Conversely, when the initial dispersion is in suspension, the product obtained is reservoir type, that is, a distinct polymeric envelope/shell encapsulating a core of dissolved active agent.

4. In the solvent extraction method wherein a physiologically active agent is dissolved or suspended into a polymer solution in an organic solvent, the resulting fluid is sprayed into a liquid of very low temperature, such as liquid argon, nitrogen or oxygen, and the organic solvents is extracted by cold ethanol from the frozen products. This method provides high loading efficiency of the drug and is applicable to active agents that lose their biological activity easily at high temperatures.

In some illustrative embodiments, the pharmaceutical formulations of the present invention are prepared by the emulsion technique. In some other embodiments, the pharmaceutical formulations of the present invention are prepared by the single emulsion technique or double emulsion technique. In yet some other embodiments, the microparticles of the present invention are prepared by the double emulsion method. In some other embodiments, the nanoparticles of the present invention are prepared by the single emulsion method.

In one embodiment, a process of preparation of the pharmaceutical formulation of griseofulvin for long-term ocular administration comprises
preparing a solution by dissolving griseofulvin and a biocompatible polymer in a suitable solvent.
mixing the griseofulvin-biocompatible polymer solution with an emulsifying agent to form a dispersion
preparing an emulsion by mixing the dispersion with water and/or aqueous solvent system comprising an emulsifier
removal of the solvents by using a suitable drying method.

In yet another embodiment of the present invention, the biocompatible polymer is PLGA.

In another embodiment, a process of preparation of the pharmaceutical formulation of griseofulvin for long-term ocular administration comprises
preparing a solution by dissolving griseofulvin or pharmaceutically acceptable salt or derivatives thereof and PLGA in DCM
mixing griseofulvin-PLGA solution to the PVA to form the dispersion and sonicating the dispersion
adding the above dispersion to water to form the W/O type of emulsion
evaporating the W/O emulsion to form the desired nanoparticles, followed by washing & freeze drying the nanoparticles.

In another embodiment, a process of preparing the pharmaceutical formulation of griseofulvin for long-term ocular administration comprises
preparing primary emulsion by mixing a) griseofulvin-biocompatible polymer solution prepared by dissolving griseofulvin and a biocompatible polymer in suitable organic solvent system and b) solution or suspension of a release modifier prepared by dissolving/dispersing the release modifier in water and/or aqueous solvent system containing an emulsifier
preparing a secondary emulsion by mixing the primary emulsion with a suitable emulsifying agent
removal of the solvents by using a suitable drying method.

In another embodiment, a process of preparing the pharmaceutical formulation of griseofulvin for long-term ocular administration comprises
preparing a solution by dissolving griseofulvin or pharmaceutically acceptable salt and PLGA in DCM
preparing a suspension by dispersing the required quantity of magnesium hydroxide in water
combined the griseofulvin-PLGA solution and magnesium hydroxide suspension to form the W/O type of emulsion by sonication process
adding PVA to the W/O emulsion and further homogenizing the formulation to get the W/O/W emulsion
evaporating the W/O/W emulsion to form the desired microparticles, followed by freeze drying the microparticles.

Examples for the suitable solvents for the polymer matrix material include but not limited to dichloromethane, chloroform, benzene, ethyl acetate, benzyl alcohol, acetone, dimethyl sulfoxide (DMSO), dimethylformamide, dimethyl acetamide, dioxane, tetrahydrofuran (THF), acetonitrile, methylene chloride, ethylene chloride, carbon tetrachloride, chloroform, lower alkyl ethers such as diethyl ether and methyl ethyl ether, hexane, cyclohexane, benzene, acetone, ethyl acetate, and the like. Examples of suitable emulsifying agents include but not limited to polyvinyl alcohol (PVA), polyethylene glycol sorbitan monolaurate (Tween), sorbitan monooleate (Span), sodium dodecyl sulphate etc.

In an embodiment, Suitable drying methods to get the microparticles or nanoparticles of the present invention include but not limited to freeze drying, spraying, rotary-evaporation, fluidized bed drying etc. In some embodiment, the solvent is removed from the microparticles or nanoparticles by solvent extraction process to get the dried particles.

According to another embodiment of the present invention, pharmaceutical formulations may include the microparticles or nanoparticles in combination with any standard physiologically and/or pharmaceutically acceptable excipients which are known in the art. The compositions should be sterile and contain a therapeutically effective amount of the microparticles or nanoparticles in a unit of weight or volume suitable for administration to a patient.

Suitable buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8.

Suitable antioxidants include but not limited to butylated hydroxytoluene, butylated hydroxy anisole, alpha-tocopherol, citric acid, ascorbic acid, monothioglycerol, sodium sulfite, sodium metabisulfite, thymol, propyl gallate, histidine, methionine, acetylcysteine, butylated hydroxy toluene, butylated hydroxy anisole, cysteine and combinations thereof. The antioxidant may be present at a range of about 0.01% w/w to about 10% w/w of the formulation.

Suitable tonicity modifying agents such as sodium chloride, dextrose, boric acid, sodium tartrate, propylene glycol, polyols (such as mannitol and sorbitol), or other inorganic or organic solutes, inorganic salts, organic salts or a combination thereof. Apart from sodium chloride, the other inorganic salts may comprise potassium chloride, magnesium chloride, calcium chloride and the organic salts may comprise conjugate base of trifluoroacetic acid.

Suitable preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

Complexing or chelating agent according to the embodiments of the present invention include but are not limited to sodium ethylene diamine tetra acetic acid (EDTA), disodium EDTA, calcium disodium EDTA, Diethylenetriaminepenta acetic acid (DTPA) or any mixtures thereof.

Surfactants according to the embodiments of the present invention include but are not limited to, glyceryl monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available TWEENS™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminate silicate, triethanolamine, polyvinyl alcohol (PVA), poloxamers, tyloxapol and polyvinylpyrrolidone (PVP). In embodiments, the preferred surfactants include, but not limited to, TWEENS™ such as TWEEN 20, TWEEN 40, TWEEN 60 or TWEEN 80, Tyloxapol, Poloxamers such as PLURONICS F68, F108, F127, and Poloxamines such as Tetronics T908.

Suspending agents according to the embodiments of the present invention include but are not limited to, cellulose derivatives, e.g., methyl cellulose, sodium carboxymethyl cellulose and hydroxypropyl methyl cellulose (HPMC), polyvinylpyrrolidone, alginates, chitosan, dextrans, gelatin, polyethylene glycols (PEG), polyoxyethylene- and polyoxypropylene ethers. In embodiments, the preferred suspending agents include, but not limited to, PEGs such as PEG 4000, PEG 6000 or PEG 8000, sodium carboxymethyl cellulose, low viscosity grade HPMCs such as viscosity grades 5 centipoises (cps), 15 cps, 50 cps or 100 cps, low viscosity grade methylcelluloses.

In one embodiment, the pharmaceutical formulations according to the present invention can be formulated as liquid form which can be administered directly to the eye or formulated as dry/lyophilized/spray-dried form, which has to be reconstituted before administration. In some embodiments, the formulations of the present invention are supplied in suitable device which includes but not limited to prefilled syringe (PFS???), Ampoule, Vial, dual chamber syringe etc.

The pharmaceutical formulations according to the present invention containing microparticles or nanoparticles may also contain a vehicle to facilitate reconstitution. Prior to administration, the microparticles or nanoparticles are suspended in a suitable vehicle for injection. The suitable quantity of vehicle used for reconstitution is ranges from about 0.05 mL to 5 mL. In one embodiment, the said vehicle is water. In another embodiment, the said vehicle is non-aqueous solvent. In another embodiment, the vehicle comprising pharmaceutical excipients includes but not limited to mannitol, sodium chloride, glucose, dextrose, sucrose, or glycerins, non-ionic surfactants (e.g. poloxamers, for example poloxamer 188, poly(oxyethylene)-sorbitan-fatty acid esters), carboxymethylcellulose sodium (CMC-Na), sorbitol, poly(vinylpyrrolidone), or aluminium monostearate in order to ensure isotonicity and to improve the wettability and sedimentation properties of the micro or nanoparticles. The wetting and viscosity enhancing agents may be present in an amount of 0.01 to 10% preferably about 0.5% to about 5% by weight in the pharmaceutical composition; the isotonicity agents are added in a suitable amount to ensure an isotonic injectable suspension. The viscosity of the diluent ranges from 0.01 cps to 20,000 cps or about 0.1 cps to about 20 cps.

The invention further provides a kit comprising the pharmaceutical formulation of the present invention in a vial, optionally equipped with a transfer set, together with a vehicle in an ampoule, vial or prefilled syringe; In another embodiment, microparticles or nanoparticles and vehicle are separated in a double chambered syringe.

In another embodiment of the present invention provides pharmaceutical formulation for long-term ocular delivery comprising griseofulvin or its pharmaceutically acceptable salts, derivatives thereof in combination with other active agents selected from group comprising N-methylprotoporphyrin (NMPP) or an analog thereof, antisense RNA targeting ferrochelatase RNA, an agent for RNA silencing or RNA interference (RNAi) targeting ferrochelatase RNA, an agent for CRISPR/Cas9-mediated or Zinc-finger nuclease-mediated genetic ablation of ferrochelatase (FECH) DNA, an agent for anti-VEGF therapy or combinations thereof.

In another embodiment of the present invention provides pharmaceutical formulation for long-term ocular delivery comprising griseofulvin or its pharmaceutically acceptable salts, derivatives thereof in combination with other active agents selected from group comprising pegaptanib, ranibizumab, aflibercept, triazolopyrimidinone or its derivatives, bevacizumab, abicipar pegol, brolucizumab, conbercept, faricimab, vorolanib, biosimilars to any of these VEGF agents, or combinations thereof wherein the formulations are used for the treatment of AMD, in particular wet-AMD.

Other non-limiting examples of active agents, any of which are suitable for use in the pharmaceutical formulations described herein include: anti-vascular endothelial growth factor therapies, i.e. anti-VEGF antibody such as anti-VEGF fragment-ranibizumab; thrombin inhibitors; antithrombogenic agents; thrombolytic agents (such as plasminogen activator, or TPA and streptokinase); fibrinolytic agents; vasospasm inhibitors; calcium channel blockers; vasodilators; antihypertensive agents; clotting cascade factors (for example, protein S); anti-coagulant compounds (for example, heparin and nadroparin, or low molecular weight heparin); antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate, minocycline, doxycycline, vancomycin, kanamycin, cephalosporins such as cephalothin, cephapirin, cefazolin, cephalexin, cephardine, cefadroxil, cefamandole, cefoxitin, cefaclor, cefuroxime, cefonicid, ceforanide, cefitaxime, moxalactam, cetizoxime, ceftriaxone, cefoperazone), geldanamycin and analogues, antifungals (such as amphotericin B and miconazole), and antivirals (such as idoxuridine trifluorothymidine, acyclovir, gancyclovir, interferon, .alpha.-methyl-P-adamantane methylamine, hydroxy-ethoxymethyl-guanine, adamantanamine, 5-iodo-deoxyuridine, trifluorothymidine, interferon, adenine arabinoside); inhibitors of surface glycoprotein receptors; antiplatelet agents (for example, ticlopidine); antimitotics; microtubule inhibitors; anti-secretory agents; active inhibitors; remodeling inhibitors; antisense nucleotides (such as morpholino phosphorodiamidate oligomer); anti-metabolites; antiproliferatives (including antiangiogenesis agents, taxol, sirolimus (rapamycin), analogues of rapamycin (“rapalogs”), tacrolimus, ABT-578 from Abbott, everolimus, paclitaxel, taxane, vinorelbine); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); mydriatics (such as atropine, cyclopentolate, homatropine, scopolamine, tropicamide, eucatropine, hydroxyamphetamine); sympathomimetics (such as epinephrine); antineoplastics (such as carmustine, cisplatin, fluorouracil); immunological drugs (such as vaccines and immune stimulants); hormonal agents (such as estrogens, estradiol, progesterol, progesterone, insulin, calcitonin, parathyroid hormone, peptide and vasopressin hypothalamus releasing factor); beta adrenergic blockers (such as timolol maleate, levobunolol HCl, betaxolol HCl); immunosuppressive agents, growth hormone antagonists, growth factors (such as epidermal growth factor, fibroblast growth factor, platelet derived growth factor, transforming growth factor beta, somatotropin, fibronectin, insulin-like growth factor (IGF)); carbonic anhydrase inhibitors (such as dichlorophenamide, acetazolamide, methazolamide); inhibitors of angiogenesis (such as angiostatin, anecortave acetate, thrombospondin, dopamine agonists; radiotherapeutic agents; peptides; proteins; enzymes; nucleic acids and nucleic acid fragments; extracellular matrix components; ACE inhibitors; free radical scavengers; chelators; antioxidants; anti-polymerases; photodynamic therapy agents; gene therapy agents; and other active agents such as prostaglandins, antiprostaglandins, prostaglandin precursors, and the like.

Certain specific aspects and embodiments of the present invention will be explained in more detail with reference to the following examples, which are provided only for purposes of illustration and should not be construed as limiting the scope of the present invention in any manner.

### EXAMPLES

Example 1: Pharmaceutical formulations of griseofulvin nanoparticle (NP) compositions with varied drug loading.

TABLE 1

| | Example | | | |
|---|---|---|---|---|
| | Example | Example | Example | Example |
| | 1A | 1B | 1C | 1D |
| Ingredients | Quantity | | | |
| Griseofulvin (mg) | 2.5 | 5 | 10 | 15 |
| Poly(lactic-co- | 97.5 | 95 | 90 | 85 |
| glycolic acid | | | | |
| (PLGA) @ (mg) | | | | |
| Poly (vinyl alcohol) | 5 | 5 | 5 | 5 |
| solution | | | | |
| (4%) (mL) | | | | |
| Dichloromethane | 0.5 | 0.5 | 0.5 | 0.5 |
| (mL) | | | | |
| Water (mL) | 30 | 30 | 30 | 30 |

@ PLGA 50:50 with a molecular weight 54-69 kDa is used.

Manufacturing process:
1) Griseofulvin and PLGA were dissolved in dichloromethane.
2) The step 1) solution was added to 4% w/w poly (vinyl alcohol) solution and sonicated for about 5 minutes to form uniform dispersion.
3) The dispersion was then added to water and stirred for about 2 hours followed by evaporation of the dichloromethane using rotavapor at 500 millibar (mbar) for about 30 minutes, thus obtained Griseofulvin-PLGA nanoparticles (GRF-PLGA-NPs).
4) Nanoparticles (NPs) were washed twice with water and collected by ultracentrifugation at 10k rcf (relative centrifugal force) for about 15 minutes.
5) Collected NPs were dispersed in 2 mL of water followed by freeze drying and stored at −80° C.
6) The obtained nanoparticles were further characterized for particle size (Z-average), polydispersity index (PDI), encapsulation efficiency (EE) and morphology of nanoparticles was estimated by SEM (Scanning Electron Microscopy) analysis. Table 2 shows the results of particle size, PDI and EE.

TABLE 2

| | Z-average | Poly Dispersity | Encapsulation |
|---|---|---|---|
| Example | (nm) | Index (PDI) | Efficiency (%) |
| Example 1A | 267.4 | 0.106 | 31.16 |
| Example 1B | 544.9 | 0.324 | 39.46 |
| Example 1C | 190.7 | 0.284 | 81.12 |
| Example 1D | 216.6 | 0.217 | 49.64 |

With varied drug loading from 2.5% to 15%, the particle size (Z-average) was observed between ˜190 to 550 nm with PDI values of 0.1 to 0.3 and encapsulation efficiency was between ˜30% to 80%. SEM images showed formation of spherical NPs with smooth surfaces, however, free drug crystals were observed with 15% drug loading.

Example 2: Pharmaceutical formulations of griseofulvin nanoparticle compositions with varied lactic acid:glycolic acid ratio of Poly(lactic-co-glycolic acid).

TABLE 3

| | Examples | |
|---|---|---|
| | Example 2A | Example 2B |
| Ingredients | Quantity | |
| Griseofulvin (mg) | 5.0 | 5.0 |
| Poly(lactic-co-glycolic acid | 95.0 | — |
| (PLGA)@ (mg) | | |
| Poly(lactic-co-glycolic acid | — | 95.0 |
| (PLGA)# (mg) | | |
| Poly (vinyl alcohol) solution | 5 | 5 |
| (1%) mL | | |
| Dichloromethane mL | 0.5 | 0.5 |
| Water mL | 30 | 30 |

@Example 2A include poly(lactic-co-glycolic acid) with 50:50 ratio of lactic acid: glycolic acid and molecular weight 54-69 kiloDaltons;
#Example 2B include poly(lactic-co-glycolic acid) with 85:15 ratio of lactic acid, glycolic acid and molecular weight 75 kiloDaltons.

Manufacturing process: Same as that of Example 1.

The nanoparticles obtained were further characterized for particle size (Z-average, nm) and polydispersity index (PDI) and encapsulation efficiency (EE), morphology through SEM analysis and in-vitro drug release profiles of griseofulvin was evaluated in phosphate buffered saline (pH 7.4) with 0.2% Tween 80 (PBST) at 37° C. Nanoparticle suspension was prepared such that the total GRF concentration was below one-third of the saturation solubility to satisfy the sink condition. 15 mL of Nanoparticle suspension was filled in a 15 mL Falcon tube and rotated vertically at 10 rpm. At predetermined time intervals, 1 mL suspension was sampled, centrifuged at 10k rcf for 10 minutes to separate 0.2 mL of the supernatant. The sampled 0.2 mL was analyzed by HPLC (mobile phase, acetonitrile:water (50:50 v/v); C18 column, Luna® 5 μm C18(2) 100 Å, 250×4.6 mm; detection wavelength, 254 nm; flow rate, 1 mL/min; injection volume, 50 μL) for determination of GRF concentration. The remainder was replenished with 0.2 mL fresh PBST, mixed well, and added back to the NP suspension for continued incubation. In vitro release testing was performed in triplicate, and results were reported as mean±standard deviation.

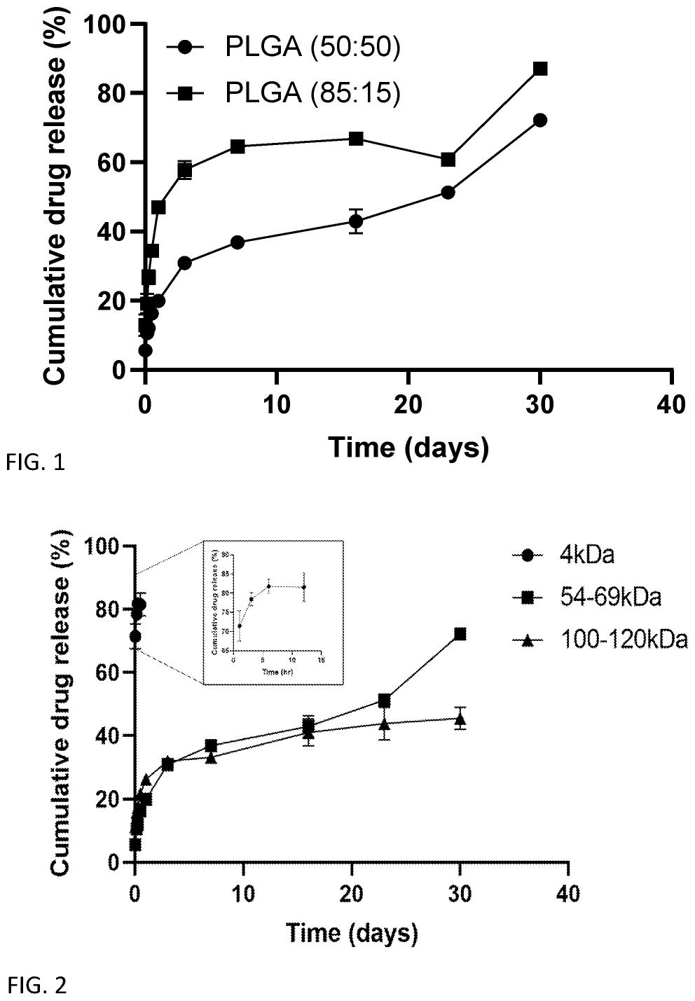

Table 4 shows the results of particle size, poly dispersity index and encapsulation efficiency and FIG. 1 shows the in-vitro release profile of griseofulvin nanoparticles.

TABLE 4

| | Z-average | Poly dispersity | Encapsulation |
|---|---|---|---|
| Example | (nm) | Index (PDI) | Efficiency (%) |
| Example 2A | 470 | 0.327 | 55.93 |
| Example 2B | 373 | 0.332 | 48.38 |

GRF-PLGA-NPs were prepared with two different LA:GA ratio (50:50 and 85:15) PLGA. NPs showed similar particle size distribution and encapsulation efficiency. SEM images showed the formation of spherical particles with a smooth surface. NPs prepared with 50:50 PLGA showed a minimal burst release (˜20% release in 1 day) followed by a continuous drug release up to 30 days.

Example 3: Pharmaceutical formulations of Griseofulvin nanoparticle compositions with varied molecular weights of PLGA.

TABLE 5

| | Examples | | |
|---|---|---|---|
| | Example | Example | Example |
| | 3A | 3B | 3C |
| Ingredients | Quantity | | |
| Griseofulvin mg | 10.0 | 5 | 10 |
| Poly(lactic-co-glycolic acid) | 90.0 | — | — |
| (50:50 4 kDa;) @ mg | | | |
| Poly(lactic-co-glycolic acid) | — | 95.0 | — |
| (50:50, 54-69 kDa) # mg | | | |
| Poly(lactic-co-glycolic acid) | — | — | 90 |
| (50:50, 100-120 kDa) $ mg | | | |
| Poly(vinyl alcohol) solution | 5 | 5 | 5 |
| (1%) mL | | | |
| Dichloromethane mL | 1 | 0.5 | 0.5 |
| Water mL | 30 | 30 | 30 |

@ Example 3A include poly(lactic-co-glycolic acid) with 50:50 ratio of lactic acid: glycolic acid and molecular weight 4 kDa (kiloDaltons).
# Example 3B include poly(lactic-co-glycolic acid) with 50:50 ratio of lactic acid, glycolic acid and molecular weight 54-69 kDa.
$ Example 3C include poly(lactic-co-glycolic acid) with 50:50 ratio of lactic acid, glycolic acid and molecular weight 100-120 kDa.

Manufacturing process: Same as that of example 1

GRF-PLGA-NPs were prepared with three different molecular weights of PLGA polymer i.e. 4 kDa (Example 3A); 54-69 kDa (Example 3B) and 100-120 kDa (Example 3C). The nanoparticles obtained were further characterized for particle size (Z-average, nm) and polydispersity index (PDI) and encapsulation efficiency (EE) and in-vitro drug release profiles of griseofulvin was evaluated in phosphate buffered saline (pH 7.4) with 0.2% Tween 80 (PBST) at 37° C. as per the procedure described in example 2 followed by determination of GRF concentration by the HPLC method described in example 2.

The long-term anti-proliferation effect of GRF loaded NPs from example 3B was studied, wherein GRF-PLGA-NPs were dispersed in 0.5 mL of growth media at GRF concentration ranging from 600 μM to 1200 μM. At specific time points (1, 2, 3, 4, 5, 6, 7, 10, 15, 20, and 25 days), tubes were centrifuged at 5k rpm for 5 minutes and the supernatant was collected and stored at −80° C. Pellet was redispersed in 0.5 mL fresh media, and the incubation was continued. A similar procedure was performed for blank-PLGA-NPs, blank-PLGA-NPs (with 5% Mg(OH)₂), GRF+blank-PLGA-NPs and unformulated GRF as well. The unformulated GRF was prepared from GRF/DMSO stock solution. The final DMSO concentration in the medium was 1%.

Human retinal endothelial cells (HREC) were plated in transparent 96 well plates at a density of 2500 cells per well in 100 μL of growth medium and incubated for 24 h at 37° C. The medium was replaced with the samples collected at different time points. After 48 hours incubation, 11.1 μL Alamar Blue reagent was added to each well. After 4 hours incubation, plates were centrifuged at 200 g for 5 minutes, and 50 μL supernatant was transferred to black 96 well plates. Fluorescence intensity of each well was read with excitation and emission wavelengths of 560 nm and 590 nm, respectively.

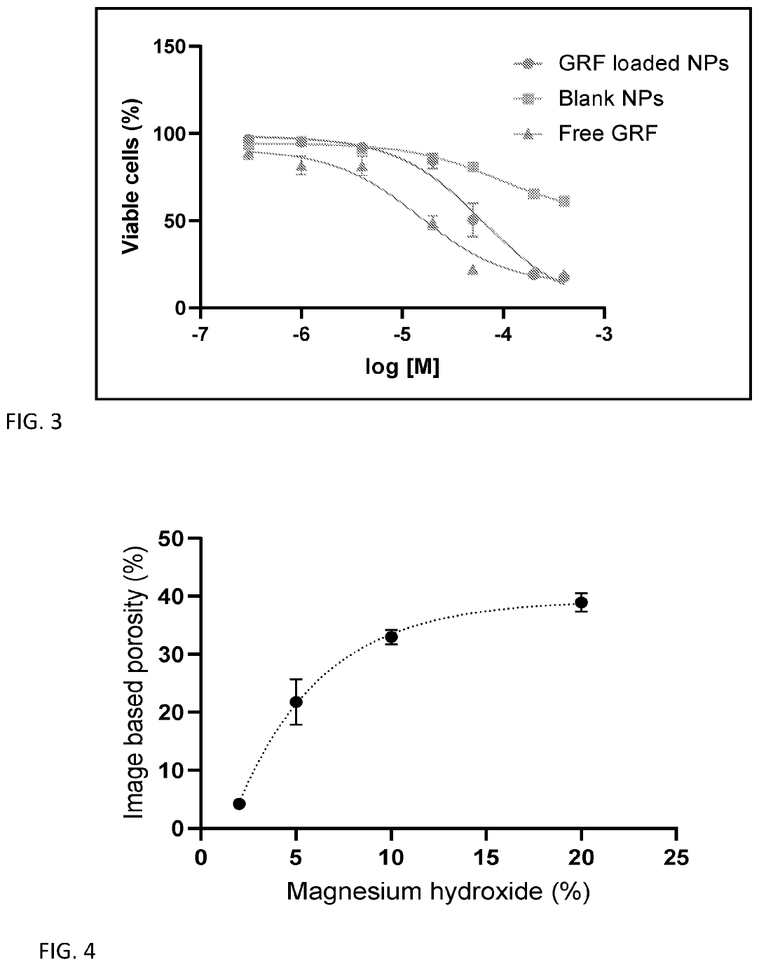

Table 6 shows the results of particle size, poly dispersity index and encapsulation efficiency of nanoparticles obtained with different molecular weight of PLGA polymer with PLGA molecular weight 4 KDa (Example 3A), 54-69 KDa (Example 3B) & 100-120 kDa (Example 3C) and FIG. 2 shows their in-vitro release profile of griseofulvin nanoparticles obtained. FIG. 3 shows the antiproliferative effect of griseofulvin loaded nanoparticles of example 3B in comparison with the free drug griseofulvin.

TABLE 6

| | Z-average | Poly dispersity | Encapsulation |
|---|---|---|---|
| Example | (nm) | Index (PDI) | Efficiency (%) |
| Example 3A | 173.3 | 0.091 | N/A |
| Example 3B | 470.1 | 0.327 | 55.93 |
| Example 3C | 718.2 | 0.229 | 74.73 |

NPs prepared with 54-69 kDa molecular weight PLGA showed a minimal burst release (˜20% release in 1 day) followed by a continuous drug release. Free GRF and GRF-loaded NPs inhibited proliferation of HRECs by 53.5% and 26.3%, respectively (at 100 μM GRF equivalent) in 48 hours, reflecting the long-term release of bioactive GRF from the NPs.

Example 4: Pharmaceutical formulations of griseofulvin microparticle (MP) compositions with varied concentrations of magnesium hydroxide [Mg(OH)₂]

TABLE 7

| | Example | | | | |
|---|---|---|---|---|---|
| | Example | Example | Example | Example | Example |
| | 4A | 4B | 4C | 4D | 4E |
| Ingredients | Quantity | | | | |
| Griseofulvin (mg) | 10 | 10 | 10 | 10 | 10 |
| Poly(lactic-co-glycolic | 90 | 88 | 85 | 80 | 70 |
| acid (PLGA) 50:50, | | | | | |
| 54-69 kDa (mg) | | | | | |
| Magnesium hydroxide | 0 | 2 | 5 | 10 | 20 |
| (mg) | | | | | |
| Poly (vinyl alcohol) | 40 | 40 | 40 | 40 | 40 |
| solution (1%) (mL) | | | | | |
| Dichloromethane (mL) | 1 | 1 | 1 | 1 | 1 |
| Water (mL) | 0.1 | 0.1 | 0.1 | 0.1 | 0.1 |

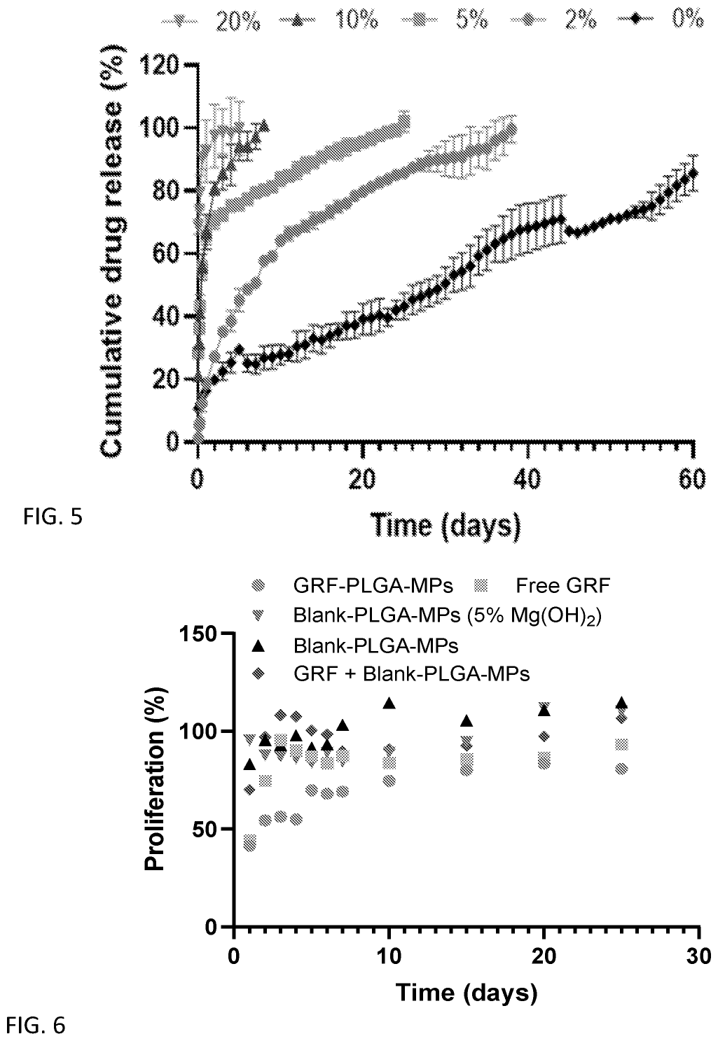

Manufacturing process
1) Drug polymer solution was preparing by dissolving griseofulvin and PLGA polymer in dichloromethane.
2) Suspension of the magnesium hydroxide was prepared by suspending magnesium hydroxide in water at different concentrations of 0% (Example 4A); 2% (Example 4B); 5% (Example 4C); 10% (Example 4D) and 20% (Example 4E)
3) The magnesium hydroxide suspension of step 2) was mixed with the drug polymer solution of step 1) and sonicated at an amplitude of 25% and a 1:1 duty cycle every 2 second for 1 minute to form a primary emulsion.
4) The primary emulsion of the step 3) was further emulsified with 40 mL of 1% PVA solution and stirred for about 2 hours to form secondary emulsion.
5) The secondary emulsion was subjected to rotary evaporation under vacuum of 50 mBar and 10 rpm for about 30 minutes.
6) The microparticles (MPs) were collected by centrifugation at 2724 rcf (relative centrifugal force) for about 5 minutes and washed thrice with deionized (DI) water. 7) The collected microparticles were subjected to freeze drying.
8) The microparticles were characterized for particle size by Mastersizer 3000; drug loading efficiency & encapsulation efficiency, porosity of the microparticles is shown in FIG. 4 (porosity was measured by image as Mean±SD from n=3 images at each point), in-vitro drug release profiles (as shown in FIG. 5) in phosphate buffered saline (pH 7.4) with 0.2% Tween 80 (PBST) at 37° C. as per the procedure described in example 2 followed by determination of GRF concentration by the HPLC method as described in example 2. Long-term proliferation assay was studied for Griseofulvin PLGA microparticles (GRF-PLGA-MPs) as per the method disclosed in example 3 and the results shown in FIG. 6. Table 8 shows the particle size, span, drug loading efficiency, encapsulation efficiency.

TABLE 8

| | | | | | Loading | Encapsulation |
|---|---|---|---|---|---|---|
| | D10 | D50 | D90 | | efficiency | efficiency |
| Example | (μm) | (μm) | (μm) | Span | (LE) % | (EE) % |
| Example 4A | 8.66 | 15.0 | 23.7 | 1.002 | 2.90 | 29.0 |
| Example 4B | 7.57 | 16.1 | 29.9 | 1.387 | 2.65 | 26.5 |
| Example 4C | 10.3 | 23.9 | 45.1 | 1.456 | 3.28 | 32.8 |
| Example 4D | 11.6 | 24.7 | 45.6 | 1.377 | 2.65 | 26.5 |
| Example 4E | 9.62 | 22.4 | 45.9 | 1.620 | 5.24 | 52.4 |

The mean particle size (D50) of the MPs was in the range of −15-25 μm and the encapsulation efficiency of the MPs was ˜29-52% (Table 4). SEM images showed spherical MPs with sizes matching those measured by laser diffraction. MPs prepared without Mg(OH)₂ had a smooth surface. MPs with Mg(OH)₂ showed porous surfaces. The porosity increased with the concentration of Mg(OH)₂. A less than 5% porosity was observed for the MPs containing 2% Mg(OH)₂. The porosity was increased to ˜21%, 32%, and 39% for the MPs containing 5%, 10%, and 20% Mg(OH)₂, respectively. It appears that the drug release from Mg(OH)₂-containing MPs is driven by the initially existing pores as well as the increasing porosity during the incubation. A higher initial porosity resulted in the high burst drug release, because of a larger effective surface area for drug diffusion.

The incorporation of Mg(OH)2 changed the GRF release profile. MPs containing 2% Mg(OH)2 showed a minimal burst release followed by continuous release. The inclusion of Mg(OH)2 in PLGA MPs led to a concentration-dependent increase in the porosity, which also grew with time. It appears that the initial pores were formed by a reaction between Mg(OH)₂ and monomers and oligomers present in PLGA as impurities to form water-soluble salts (e.g., Mg(OH)₂+CH₃—CH(OH)—COOH (lactic acid)=Mg((OCO—CH(OH)—CH₃)₂ (magnesium lactate)) and the pores increased during the incubation when PLGA started to degrade and the encapsulated Mg(OH)₂ reacted with the degradation products (e.g., Mg(OH)₂+PLGA-COOH=Mg(OCO-PLGA)₂).

GRF-PLGA-MPs inhibited the proliferation of HREC throughout the incubation period, whereas unformulated GRF and a mixture of unformulated GRF and blank PLGA MPs were effective only at the first time point indicating that the GRF release from GRF-PLGA-MPs led to prolonged anti-proliferative effects on HREC. Blank PLGA MPs showed no effect on HREC proliferation. To compare the treatments in the sustained anti-proliferative effect on HREC, time averaged proliferation inhibition index (PII) was calculated. FIG. 6 shows the PII calculation for GRF-PLGA-MPs. GRF-PLGA-MPs showed higher PII (24.7) than unformulated GRF (13.6) (numbers in the box in FIG. 6 represents PII), indicating that the GRF release from GRF-PLGA-MPs led to prolonged anti-proliferative effects on HREC.

Example 5: Pharmaceutical formulations of Griseofulvin microparticle (MP) compositions with varied concentrations of magnesium phosphate [Mg₂(PO₄)₃].

TABLE 9

| | Examples | | | | |
|---|---|---|---|---|---|
| | Example | Example | Example | Example | Example |
| | 5A | 5B | 5C | 5D | 5E |
| Ingredients | Quantity | | | | |
| Griseofulvin (mg) | 10 | 10 | 10 | 10 | 10 |
| Poly(lactic-co- | 90 | 88 | 85 | 80 | 70 |
| glycolic acid 50:50, | | | | | |
| 54-69 kDa (mg) | | | | | |
| Magnesium phosphate | 0 | 2 | 5 | 10 | 20 |
| (mg) | | | | | |
| Poly (vinyl alcohol) | 40 | 40 | 40 | 40 | 40 |
| solution (1%) (mL) | | | | | |
| Dichloromethane (mL) | 1 | 1 | 1 | 1 | 1 |
| Water (mL) | 0.1 | 0.1 | 0.1 | 0.1 | 0.1 |

Manufacturing process: Same as that of example 4

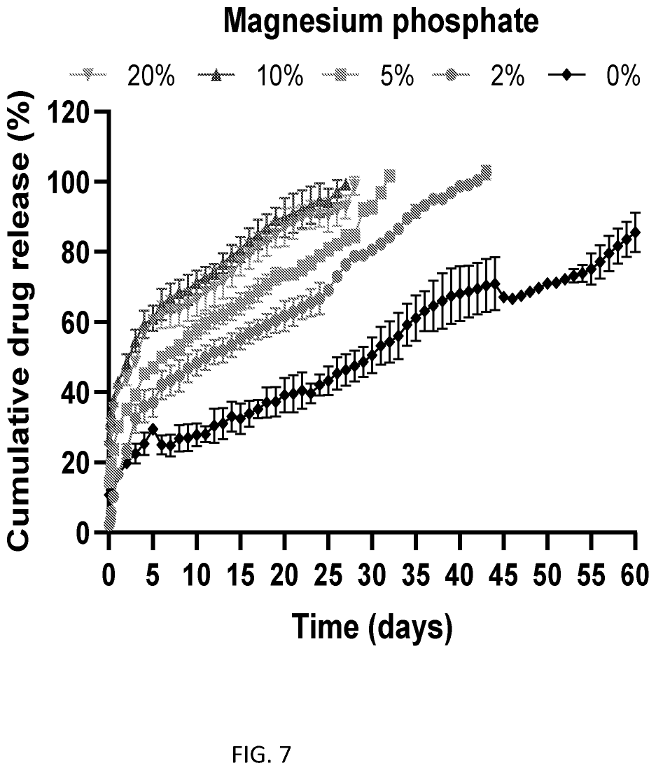

The microparticles were characterized for in-vitro drug release profiles in phosphate buffer saline (pH 7.4) with 0.2% Tween 80 (PBST) at 37° C. as shown in FIG. 7. Mg2(PO4)3 is a relatively weaker base than Mg(OH)₂, which was expected to have less interaction with PLGA impurities or degradation products and, hence, relatively a mild effect on initial and subsequent release.

In vitro drug release of the MPs was evaluated in the same conditions as described in example 2 (i.e PBST, pH 7.4, at 37° C., falcon tube method and HPLC determination of GRF concentration). MPs with 5% Mg₂(PO₄)₃ showed ˜30% release in 1 day followed by a slow release. A lower burst (˜15%) and slow drug release were observed with MPs containing 2% Mg₂(PO₄)₃. MPs with Mg₂(PO₄)₃ also showed porous surfaces, but the porosity of Mg₂(PO₄)₃-containing MPs was lower than the Mg(OH)₂-containing MPs at comparable concentrations. The porosity increased with the concentration of Mg₂(PO₄)₃ and with the duration of incubation. Mg₂(PO₄)₃ showed an overall less impact on the porosity and the release of GRF from MPs than Mg(OH)₂.

Example 6: Pharmaceutical formulations of Griseofulvin microparticle (MP) compositions with varied concentrations of sodium chloride.

TABLE 10

| | Examples | | | | |
|---|---|---|---|---|---|
| | Example | Example | Example | Example | Example |
| | 6A | 6B | 6C | 6D | 6E |
| Ingredients | Quantity | | | | |
| Griseofulvin (mg) | 10 | 10 | 10 | 10 | 10 |
| Poly(lactic-co-glycolic | 90 | 88 | 85 | 80 | 70 |
| acid 50:50, 54-69 | | | | | |
| kDa (mg) | | | | | |
| Sodium chloride (mg) | 0 | 2 | 5 | P0 | 20 |
| Poly (vinyl alcohol) | 40 | 40 | 40 | 40 | 40 |
| solution (1%) | | | | | |
| Dichloromethane (mL) | 1 | 1 | 1 | 1 | 1 |
| Water (mL) | 0.1 | 0.1 | 0.1 | 0.1 | 0.1 |

Manufacturing process: Same as that of example 4.

Those skilled in the art will recognize that numerous modifications can be made to the specific implementations described above. The implementations should not be limited to the particular limitations described. Other implementations may be possible. While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A pharmaceutical formulation for ocular administration comprising griseofulvin, or its pharmaceutically acceptable salts or derivatives thereof, and at least one biocompatible polymer, wherein the formulation exhibits a long-term release of griseofulvin for a period of about 1 month to about 6 months.

2. The pharmaceutical formulation according to claim 1, wherein the formulation exhibits long-term release of griseofulvin for a period of about 1 month to about 3 months.

3. The pharmaceutical formulation according to claim 2, wherein the formulation exhibits long-term release of griseofulvin for a period of about 1 month.

4. The pharmaceutical formulation according to claim 3, wherein the formulation exhibits an in vitro griseofulvin release profile of
less than about 40% release within 1 day; about 40% to about 70% release within 10 days; and more than about 70% release within 30 days.

5. The pharmaceutical formulation according to claim 4, wherein the formulation exhibits an in vitro griseofulvin release profile of
less than about 30% release within 1 day; about 40% to about 70% release within 10 days; and more than about 80% release within 30 days.

6. The pharmaceutical formulation according to claim 5, wherein the formulation exhibits an in vitro griseofulvin release profile of
less than about 20% release within 1 day; about 40% to about 70% release within 10 days; about 70% to about 85% release within 20 days; and more than about 85% release within 30 days.

7. The pharmaceutical formulation according to claim 1, wherein the biocompatible polymer is selected from the group comprising poly(lactides), poly(glycolides), poly(lactide-co-glycolides), poly(lactic acid)s, poly(glycolic acid)s, poly(lactic acid-co-glycolic acids)s, polycaprolactones, polycarbonates, polyesteramides, polyanhydrides, poly(amino acids), polyorthoesters, polycyanoacrylates, poly(p-dioxanone)s, poly(alkylene oxalate)s, biodegradable polyurethanes, blends and copolymers thereof.

8. The pharmaceutical formulation according to claim 7, wherein the biocompatible polymer is poly(lactic acid-co-glycolic acid) polymer.

9. The pharmaceutical formulation according to claim 8, wherein the poly(lactic acid-co-glycolic acid) polymer has an average molecular weight range from about 1,000 to about 150,000 Daltons.

10. The pharmaceutical formulation according to claim 9, wherein the poly(lactic acid-co-glycolic acid) polymer has an average molecular weight range from about 40,000 to about 75,000 Daltons.

11. The pharmaceutical formulation according to claim 8, wherein the poly (lactic acid-co-glycolic acid) polymer has a molar ratio of lactic acid to glycolic acid range from about 90:10 to about 10:90.

12. The pharmaceutical formulation according to claim 11, wherein the poly (lactic acid-co-glycolic acid) polymer has a molar ratio of lactic acid to glycolic acid range from about 60:40 to about 40:60.

13. The pharmaceutical formulation according to claim 8, wherein the poly (lactic acid-co-glycolic acid) polymer concentration is about 70% to about 99% by weight relative to the total weight of the formulation.

14. The pharmaceutical formulation according to claim 1, further comprising a release modifier selected from the group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran.

15. The pharmaceutical formulation according to claim 14, wherein the release modifier is magnesium hydroxide

16. The pharmaceutical formulation according to claim 14, wherein the release modifier is magnesium phosphate

17. The pharmaceutical formulation according to claim 14, wherein the release modifier concentration is about 0.25% to about 30% by weight relative to the total weight of the formulation.

18. The pharmaceutical formulation according to claim 1, wherein griseofulvin is in the form of microparticles or nanoparticles or a combination thereof.

19. (canceled)

20. The pharmaceutical formulation according to claim 18, wherein the mean particle size of griseofulvin microparticles ranges from about 5 μm to about 100 μm or the mean particle size of griseofulvin nanoparticles ranges from about 10 nm to about 1000 nm.

21. The pharmaceutical formulation according to claim 20, wherein the mean particle size of griseofulvin microparticles ranges from about 5 μm to about 50 μm or the mean particle size of griseofulvin nanoparticles ranges from about 100 nm to about 600 nm.

22. (canceled)

23. The pharmaceutical formulation according to claim 18, wherein the porosity of griseofulvin microparticles is less than about 50% or the porosity of griseofulvin nanoparticles is less than about 50%.

24. The pharmaceutical formulation according to claim 18, wherein the span value of griseofulvin microparticles is about 0.5 to about 5.

25. The pharmaceutical formulation according to claim 18, wherein the weight ratio of griseofulvin to biocompatible polymer in the microparticles ranges from about 1:3 to about 1:50.

26. A pharmaceutical formulation for a long-term ocular administration of griseofulvin comprising
a) griseofulvin, or its pharmaceutically acceptable salts or derivatives thereof, at a concentration of about 0.5% to about 25% by weight of the formulation;
b) a biocompatible polymer at a concentration of about 70% to about 99% by weight of the formulation; and
c) a release modifier at a concentration of about 0.25% to about 30% by weight of the formulation.

27. The pharmaceutical formulation according to claim 26, wherein the biocompatible polymer is poly(lactic acid-co-glycolic acid) polymer and the release modifier is magnesium hydroxide or magnesium phosphate.

28. (canceled)

29. The pharmaceutical formulation according to claim 27, wherein the formulation exhibits long-term release of griseofulvin for a period of about 1 month to about 3 months.

30. The pharmaceutical formulation according to claim 29, wherein the formulation exhibits long-term release of griseofulvin for a period of about 1 month.

31. The pharmaceutical formulation according to claim 30, wherein the formulation exhibits an in vitro griseofulvin release profile of
a) less than about 40% release within 1 day;
b) about 40% to about 70% release within 10 days; and
c) more than about 70% release within 30 days.

32. The pharmaceutical formulation according to claim 30, wherein the formulation exhibits an in vitro griseofulvin release profile of
a) less than about 20% release within 1 day;
b) about 40% to about 70% of release within 10 days;
c) about 70% to about 85% release within 20 days; and
d) more than about 85% release within 30 days.

33. (canceled)

34. (canceled)

35. (canceled)

36. (canceled)

37. (canceled)

38. The pharmaceutical formulation according to claim 18, wherein the polydispersity index of griseofulvin nanoparticles is about 0.1 to about 1.0.

39. The pharmaceutical formulation according to claim 18, wherein the weight ratio of griseofulvin to biocompatible polymer in the nanoparticles ranges from about 1:3 to about 1:50.

40. A process of preparing a pharmaceutical formulation of griseofulvin for long-term ocular administration comprising
a) preparing a solution by dissolving griseofulvin and a biocompatible polymer in a suitable solvent;
b) mixing the griseofulvin-biocompatible polymer solution with an emulsifying agent to form a dispersion;
c) preparing an emulsion by mixing the dispersion with water and/or an aqueous solvent system containing an emulsifier; and
d) removing the solvents by using a suitable drying method.

41. A process of preparing a pharmaceutical formulation of griseofulvin for long-term ocular administration comprising
a) preparing a primary emulsion by mixing a) a griseofulvin-biocompatible polymer solution prepared by dissolving griseofulvin and a biocompatible polymer in a suitable organic solvent system and b) a solution or a suspension of a release modifier prepared by dissolving/dispersing a release modifier in water and/or an aqueous solvent system containing an emulsifier;
b) preparing a secondary emulsion by mixing the primary emulsion with an aqueous solution containing a suitable emulsifying agent; and
c) removing the solvents by using a suitable drying method.

42. The process according to claim 40, wherein the biocompatible polymer is poly(lactic acid-co-glycolic acid) polymer and the release modifier is selected from group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran.

43. The process according to claim 40, wherein the suitable solvent is selected from the group comprising dichloromethane, acetonitrile, tetrahydrofuran, ethylacetate, chloroform, acetone, and hexafluoroisopropanol.

44. The process according to claim 40, wherein the suitable emulsifying agent is selected from the group comprising polyvinyl alcohol (PVA), non-ionic surfactants (such as Poloxamers, Tweens), anionic surfactants (such as sodium oleate, sodium stearate or sodium lauryl sulfate), gelatin, polyvinylpyrrolidone, carboxymethyl cellulose and its derivatives.

45. (canceled)

46. (canceled)

47. (canceled)

48. (canceled)

49. A method for the treatment of an eye disease of a patient comprising the step of administrating a therapeutically effective amount of a pharmaceutical formulation of claim 1.

50. (canceled)

51. The method according to claim 49, wherein said eye disease is selected from the group consisting of retinopathy of prematurity (ROP), proliferative diabetic retinopathy (PDR), diabetic retinopathy, wet age-related macular degeneration (AMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and combinations thereof.

52. The method of claim 49, wherein the pharmaceutical formulation is administered by intravitreal injection, suprachoroidal injection, subretinal injection, intraocular injection, periocular injection, intra-bulbar injection, intracameral injection, sub-tenon injection, subconjunctival injection, an ocular insert, or an implant.

53. The method of claim 49, wherein the pharmaceutical composition is administered by intravitreal injection.

54. The process according to claim 41, wherein the biocompatible polymer is poly(lactic acid-co-glycolic acid) polymer and the release modifier is selected from group comprising magnesium hydroxide, magnesium phosphate, magnesium carbonate, zinc carbonate, zinc phosphate, zinc hydroxide, calcium hydroxide, calcium carbonate, calcium phosphate, tetramethylammonium hydroxide, polyethylene glycol, poloxamer, polyvinylpyrrolidone, sodium chloride, magnesium chloride, sucrose, trehalose, cyclodextrins, and dextran.

55. The process according to claim 41, wherein the suitable solvent is selected from the group comprising dichloromethane, acetonitrile, tetrahydrofuran, ethylacetate, chloroform, acetone, and hexafluoroisopropanol.

56. The process according to claim 41, wherein the suitable emulsifying agent is selected from the group comprising polyvinyl alcohol (PVA), non-ionic surfactants (such as Poloxamers, Tweens), anionic surfactants (such as sodium oleate, sodium stearate or sodium lauryl sulfate), gelatin, polyvinylpyrrolidone, carboxymethyl cellulose and its derivatives.
